(19) United States
(12) Patent Application Publication
(10) Pub. No.: US 2025/0360184 A1
(43) Pub. Date: Nov. 27, 2025

(54) METHODS FOR TREATING ACUTE CONDITIONS USING LIPID BINDING PROTEIN-BASED COMPLEXES

(71) Applicants: Cyrille TUPIN, Balma (FR); Constance PEYROTTES, Balma (FR); Ronald BARBARAS, Balma (FR); Stanislas FAGUER, Balma (FR); ABIONYX PHARMA SA, Balma (FR)

(72) Inventors: Cyrille TUPIN, Castelmaurou (FR); Constance PEYROTTES, Le Castéra (FR); Ronald BARBARAS, Seilh (FR); Stanislas FAGUER, Toulouse (FR)

(21) Appl. No.: 18/872,646

(22) PCT Filed: Jun. 9, 2023
(86) PCT No.: PCT/IB2023/000513

§ 371 Date: Dec. 6, 2024

Related U.S. Application Data
(60) Provisional application No. 63/351,125, filed on Jun. 10, 2022.

(51) Int. Cl. A61K 38/17; A61P 11/00; A61P 29/00

(52) U.S. Cl. CPC A61K 38/1709; A61P 11/00; A61P 29/00

(57) ABSTRACT

Methods for treating acute conditions (e.g., acute conditions comprising acute inflammation), such as cytokine release syndrome, sepsis and acute kidney injury using lipid binding protein-based complexes.

### 1. CROSS-REFERENCE TO RELATED APPLICATIONS

This application claims the priority benefit of U.S. application No. 63/351,125, filed Jun. 10, 2022, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Jun. 1, 2023 is named CRN-049WO_SL.xml and is 3,263 bytes in size.

### 3. BACKGROUND

Various acute conditions, for example, conditions that can be associated with acute inflammation such as sepsis, acute kidney injury (AKI), and cytokine release syndrome (CRS) are common and potentially life threatening. Current treatments for such conditions are oftentimes inadequate or suboptimal.

### 3.1. Sepsis

Sepsis is a potentially life-threatening systemic response of the immune system that arises from infection and which can cause injury to tissues and organs (Singer et al., 2016, JAMA. 315(8):801-10). Common signs and symptoms of sepsis include fever, increased heart rate, increased breathing rate, and confusion. There may also be symptoms related to a specific infection, such as a cough with pneumonia, or painful urination with a kidney infection (Jui et al., 2011, “Ch. 146: Septic Shock.” In Tintinalli J E, et al. (eds.). Tintinalli's Emergency Medicine: A Comprehensive Study Guide (7th ed.). New York: McGraw-Hill. pp. 1003-14). Severe sepsis can be associated with poor organ function or blood flow (Dellinger et al., 2013, Critical Care Medicine. 41(2):580-637). The presence of low blood pressure, high blood lactate, or low urine output may suggest poor blood flow. Sepsis can progress to septic shock, which is characterized by low blood pressure that does not improve after fluid replacement (Dellinger et al., 2013, Critical Care Medicine. 41(2):580-637).

Bacterial infections are the most common cause of sepsis, but fungal, viral, and protozoan infections can also lead to sepsis (Jui et al., 2011, “Ch. 146: Septic Shock.” In Tintinalli J E, et al. (eds.). Tintinalli's Emergency Medicine: A Comprehensive Study Guide (7th ed.). New York: McGraw-Hill. pp. 1003-14). Common locations for the primary infection include the lungs, brain, urinary tract, skin, and abdominal organs (Jui et al., 2011, “Ch. 146: Septic Shock.” In Tintinalli J E, et al. (eds.). Tintinalli's Emergency Medicine: A Comprehensive Study Guide (7th ed.). New York: McGraw-Hill. pp. 1003-14). Risk factors include being very young, older age, a weakened immune system from conditions such as cancer or diabetes, major trauma, or burns (cdc.gov/sepsis/what-is-sepsis.html). A sepsis diagnosis can be based on the sequential organ failure assessment score (SOFA score) (Vincent et al. 1996, Intensive Care Med, 22:707-710). A sepsis diagnosis can also be based on the shortened SOFA score, also known as the quick SOFA score (qSOFA), which requires at least two of the following three: increased breathing rate, change in the level of consciousness, and low blood pressure (Singer et al., 2016, JAMA. 315(8):801-10). For example, a diagnosis of sepsis can be based on an increase in a subject's total SOFA score or qSOFA score.

Sepsis can require immediate treatment with intravenous fluids and antimicrobials (Rhodes et al., 2017, Intensive Care Medicine. 43(3):304-377). Ongoing care often continues in an intensive care unit. If an adequate trial of fluid replacement is not enough to maintain blood pressure, then the use of medications that raise blood pressure can become necessary. Mechanical ventilation and dialysis may be needed to support the function of the lungs and kidneys, respectively. Other helpful measurements include cardiac output and superior vena cava oxygen saturation (Dellinger et al., 2013, Critical Care Medicine. 41(2):580-637).

The risk of death from sepsis is as high as 30%, while for severe sepsis it is as high as 50%, and septic shock 80% (Jawad et al., 2012, J Glob Health. 2(1):010404). Early detection and treatment is essential for survival and limiting disability.

### 3.2. Acute Kidney Injury

Acute kidney injury (AKI) is a common occurrence in ICU patients, with an estimated incidence of >50% (Hoste et al., 2015, Intensive Care Med; 41:1411-1423). Furthermore, increasing AKI severity is associated with increased mortality. Sepsis is the major cause of AKI, accounting for 45% to 70% of cases, and approximately 25% of sepsis is of intra-abdominal origin (Seymour et al., 2016, JAMA, 315:762-774; Bagshaw et al., 2007, Clin J Am Soc Nephrol, 2:431-439). Ischemia/reperfusion injury (IRI) can cause AKI and is a common complication in subjects receiving an organ transplant, with an incidence of 50-75% after lung and heart transplantation (Gueler et al., 2014, Transplantation 98:337-338. Cardiac surgery associated AKI (CSA AKI) has been reported to occur in up to 30% of subjects who undergo cardiac surgery (Rosner and Okusa, 2006, Clin J Am Soc Nephrol. 1(1):19-323). Post-surgical IL6 and IL10 levels are predictive of AKI development and outcome (Zhang et al., 2015, J Am Soc Nephrol. 26(12):3123-32) and there are no good treatment options other than dialysis (Küllmar et al., 2020, Crit Care Clin. 36(4):691-704.

Early diagnosis of AKI in the setting of sepsis is important in order to provide optimal treatment and avoid further kidney injury (Peerapornratana et al., 2019, Kidney International 2019, 96:1083-1099). Treatment options for sepsis-related AKI are limited to supportive care. The use of blood filtration devices, including high volume hemofiltration and polymyxin B hemoperfusion, have not shown significant benefit (Joannes-Boyau et al., 2013, Intensive care medicine, 39:1535-1546; Zhang et al., 2012, Nephrology, dialysis, transplantation: official publication of the European Dialysis and Transplant Association—European Renal Association 27:967-973; Vincent et al., 2005, Shock, 23:400-405; Cruz et al., 2009, JAMA, 301:2445-2452; Payen et al., 2015, Intensive care medicine, 41:975-98; Dellinger et al., 2018, JAMA, 320:1455-463).

Experimental pharmacologic treatments are usually targeted for AKI rather than sepsis-induced AKI, with the exception of alkaline phosphatase (AP), angiotensin II (ATII), levocarnitine and reltecimod (AB103). In a recent clinical trial, recombinant AP did not reduce endogenous creatinine clearance, the primary clinical endpoint, but did improve mortality, which was a secondary endpoint (Pickkers et al., 2018, JAMA, 320:1998-2009). ATII showed some benefit in a post-hoc analysis of AKI patients in a high-output shock study (ATHOS-3) and is currently being study in sepsis-related AKI in the ASK-IT trial (NCT00711789), however no updates have been given since 2011. Levocarnitine did not show organ dysfunction improvement in septic shock in the RACE study (Jones et al., 2018, JAMA network open, 1:e186076) but is currently being studied as an adjunct treatment for septic shock patients with AKI in the Carnisave trial (NCT02664753). Reltecimod was being studied in a Phase 3 placebo-controlled trial in patients with sepsis-associated AKI (NCT03403751), but the study was recently terminated due to slow enrollment (clinicaltrials.gov/ct2/show/NCT03403751).

Alterations in lipid and lipoprotein metabolism have been reported to occur during infection leading to a redistribution of nutrients to cells that are important in host defense or tissue repair (Khovidhunkit et al., 2004, J Lipid Res, 45(7):1169-96). In addition, lipoproteins and lipids play a key role in host defense against infection and protect the host from the toxic effects of microorganisms (Feingold and Grunfeld, 2012, J Lipid Res. 53(12):2487-248). High-density lipoprotein (HDL) is a key component of circulating blood and mainly contains phospholipids, free cholesterol, cholesteryl ester, triglycerides, apolipoproteins (Apo A-I, Apo A-II), and other proteins. It is considered an anti-inflammatory lipoprotein, which regulates vascular endothelial function and immunity (Singh et al., 2007, JAMA, 298(7):786-798; Navab et al., 2011, Nat Rev Cardiol 8(4):222-32). Indeed, HDL plays pivotal protective roles in all the steps of endothelial dysfunction, including suppression of inflammatory signaling in immune effector cells and direct inhibition of endothelial activation. Clinical studies have demonstrated that HDL levels drop by 40-70% during systemic inflammation and it is associated with a poor prognosis in septic subjects (van Leeuwen et al., 2003, Critical care medicine, 31:1359-1366; Chien et al., 2005, Critical care medicine, 33:1688-1693; Tsai et al., Journal of hepatology, 50:906-915; Eggesbø et al., 1996, Cytokine, 8(2):152-160; Morin et al., 2015, Frontiers in Pharmacology, doi.org/10.3389/fphar.2015.00244). Moreover, low levels of HDL have been associated with increased risk of acute kidney injury (AKI) in course of sepsis (Roveran et al., 2017, Journal of internal medicine, 281:518-529; Zhang et al., 2009, Am J Physiol Heart Circ Physiol 297:H866-H873). Renal function and plasma HDL are strongly related to each other as kidneys are implicated in the recycling of senescent HDL particles and their filtration function is associated with their levels and contents (Yang et al., 2016, Current opinion in nephrology and hypertension, 25:174-179).

Treatments based on HDL have been proposed for sepsis-induced systemic inflammatory reaction syndrome (Morin et al., 2015, Frontiers in Pharmacology, doi.org/10.3389/fphar.2015.00244; Tanaka et al., 2020, Crit Care 24:134). Several studies have suggested that the correction of dyslipoproteinemia by recombinant HDL (rHDL) may offer a strategy for the prevention and treatment of systemic inflammatory response (Morin et al., 2015, Frontiers in Pharmacology, doi.org/10.3389/fphar.2015.00244; Roveran et al., 2017, Journal of internal medicine, 281:518-529; Pajkrt et al., 1996, Journal of Experimental Medicine, 184(5): 1601-1608; Pajkrt et al., 1997, Thrombosis and Haemostasis 77(2):303-7; Guo et al., 2013, J. Biol. Chem. 288(25):17947-53; Li et al., 2008, European journal of pharmacology 590:417-422; McDonald et al., 2003, Shock 20(6):551-7). CSL-111, a rHDL originally produced for atherosclerosis treatment (Tardif et al., 2007, JAMA, 297(15):1675-82), has shown efficacy in reducing the inflammatory response during LPS-induced endotoxemia in vitro and in rabbit (Casas et al., 1995, The Journal of surgical research, 59:544-552) and human models (Pajkrt et al., 1996, Journal of Experimental Medicine, 184(5):1601-8; Pajkrt et al., 1997, Thrombosis and Haemostasis, 77(2):303-7). In a human model, the infusion of CSL-111 has been shown to decrease the procoagulant state caused by endotoxin exposure, reduce monocyte activation and cytokine production and ameliorate clinical symptoms (Pajkrt et al., 2016, Journal of Experimental Medicine, 184(5): 1601-1608; Pajkrt et al., 1997, Thrombosis and Haemostasis, 77(2):303-7). ApoA1 Milano, a naturally variant of ApoA1, was widely studied in the context of cardiovascular disease (CVD) in a Phase I trial (Casas et al., 1995, The Journal of surgical research, 59:544-552) and other further clinical studies. Recently, Zhang and colleagues demonstrated that ApoA1 was also efficacious against inflammation in an endotoxemic rat model (Zhang et al., 2015, Biological Chemistry, 396(1):53-60). Among HDL mimetic peptides, L-4F has been employed in several preclinical model of sepsis and has been shown to block production of cytokines, reverse sepsis-induced hypotension, prevent organ damage, and restore renal, hepatic, and cardiac function, and increase survival rate (Zhang et al., 2009, Am J Physiol Heart Circ Physiol 297: H866-H873). The altered serum lipid levels, especially cholesterol level, have been reported to occur also during infection with viruses (Meher et al., 2019, J. Phys. Chem. B, 123(50):10654-10662) including human immunodeficiency virus (HIV) and hepatitis C virus (HCV). Despite the interest in HDL and HDL therapeutics, no HDL or HDL mimetic has received regulatory approval for the treatment of sepsis or AKI, including sepsis-related AKI, ischemia/reperfusion AKI and CSA AKI.

### 3.3. Cytokine Release Syndrome

Cytokine release syndrome (CRS), also called cytokine storm syndrome (CSS), is a systemic inflammatory response that can be caused by a variety of factors such as infection or treatment with some types of immunotherapy, such as monoclonal antibodies and adoptive T-cell therapies (Shimabukuro-Vornhagen, et al., 2018, J. Immunotherapy Cancer, 6:56). Symptoms of CRS include fever, nausea, headache, rash, rapid heartbeat, low blood pressure, and trouble breathing. Most patients with CRS have a mild reaction, but sometimes CRS can be severe and even life threatening (NCI Dictionary of Cancer Terms (cancer.gov/publications/dictionaries/cancer-terms/def/cytokine-release-syndrome)).

Since late 2019, a novel coronavirus, COVID-19 (SARS-CoV-2), has been spreading around the globe. Data suggest that there are mild or severe cytokine storms in severely affected patients, accompanied by high expression of interleukin-6 (IL-6). CRS may contribute to death of these patients (Zhang et al., 2020, International Journal of Antimicrobial Agents, doi.org/10.1016/j.ijantimicag.2020.105954; Mehta et al., 2020, The Lancet, 395(10229):1033-1034).

Thus, there remains a need for new treatments for acute conditions such as sepsis, AKI, including sepsis related AKI, ischemia/reperfusion AKI and CSA AKI, and CRS, for example CRS associated with immunotherapy and CRS secondary to infections such as COVID-19.

### 4. SUMMARY

The present disclosure provides methods for treating subjects with acute conditions, for example conditions associated with acute inflammation, with a high dose of a lipid binding protein-based complex (e.g., administered as a formulation comprising a lipid binding protein such as ApoA-I and one or more lipids). The high dose is typically higher than a dose that would be used to treat a chronic condition, such as familial hypercholesterolemia. The high dose is typically administered over a relatively short period of time, for example, over a period of one day to two weeks, and typically comprises multiple administrations of the lipid binding protein-based complex, for example two to 10 individual doses. The individual doses can be separated by less than one day (e.g., twice daily administration), or one day or more (e.g., once daily administration).

In some embodiments of the methods of the disclosure, the lipid binding protein-based complex comprises a sphingomyelin and/or a negatively charged lipid, for example CER-001. CER-001 is a negatively charged lipoprotein complex, and comprises recombinant human ApoA-I, sphingomyelin (SM), and 1, 2-dihexadecanoyl-sn-glycero-3-phospho-(1′-rac-glycerol) (Dipalmitoylphosphatidyl-glycerol; DPPG). It mimics natural, nascent discoidal pre-beta HDL, which is the form that HDL particles take prior to acquiring cholesterol. Without being bound by theory, it is believed that CER-001 therapy can reduce serum levels of inflammatory cytokines such as IL-6, thereby providing a clinical benefit to subjects having an acute condition or at risk of an acute condition, for example subjects having or at risk of an acute inflammatory condition.

In some aspects, the present disclosure provides methods for treating subjects with an infection, for example a coronavirus infection such as COVID-19, with lipid binding protein-based complexes (e.g., CER-001).

In some aspects, the present disclosure provides methods for treating subjects with sepsis and methods for treating subjects with AKI or at risk for AKI with lipid binding protein-based complexes (e.g., CER-001).

In one aspect, the disclosure provides a method of treating a subject with sepsis, comprising administering to the subject a lipid binding protein-based complex (e.g., CER-001).

In another aspect, the disclosure provides a method of treating a subject with acute kidney injury (AKI) or at risk for AKI (e.g., a subject with sepsis which has not yet caused AKI, an organ transplant recipient, or a subject who has undergone cardiac surgery, or a subject having acute or chronic liver disease and at risk of hepatorenal syndrome (HRS)), comprising administering to the subject a lipid binding protein-based complex (e.g., CER-001).

In some aspects, the disclosure provides methods for treating cytokine release syndrome (CRS) and/or reducing one or more inflammatory markers in a subject in need thereof with a lipid binding protein-based complex (e.g., CER-001).

In one aspect, the disclosure provides methods of treating a subject with CRS or at risk of CRS, e.g., a subject with CRS secondary to COVID-19 or a subject with CRS caused by immunotherapy, comprising administering a therapeutically effective amount of a lipid binding protein-based complex (e.g., CER-001) to the subject.

In another aspect, the disclosure provides methods of reducing serum levels of one or more inflammatory markers, e.g., one or more markers associated with CRS such as IL-6, in a subject in need thereof. The subject can be, for example, a subject with CRS or a subject at risk of CRS, for example a subject infected with a virus such as COVID-19 or a subject receiving immunotherapy.

In some aspects, the present disclosure provides dosing regimens for lipid binding protein-based therapy (e.g., CER-001 therapy) for subjects with an acute condition (e.g., associated with acute inflammation), for example sepsis, AKI (e.g., AKI caused by sepsis, ischemia/reperfusion, cardiac surgery, or hepatorenal syndrome), or at risk for an acute condition such as AKI (e.g., a subject with sepsis which has not yet led to AKI) or CRS.

The dosing regimens of the disclosure typically entail multiple administrations of CER-001 to a subject (e.g., administered daily or twice in one day). The CER-001 therapy can be continued for a pre-determined period, e.g., for one week or less (e.g., one day, two days, three days, four days, five days, six days, or seven days) or a period longer than one week (e.g., two weeks). Alternatively, administration of CER-001 to a subject can be continued until one or more symptoms of the acute condition (e.g., acute inflammation or CRS) are reduced or continued until the serum levels of one or more inflammatory markers are reduced, for example reduced to a normal level or reduced relative to a baseline measurement taken prior to the start of CER-001 therapy. For subjects at risk of CRS or AKI due to an infection or at risk of CRS due to immunotherapy, the therapy can in some embodiments be continued until the subject has recovered from the infection or discontinues immunotherapy.

The dosing regimens of the disclosure can entail administering a lipid binding protein-based complex (e.g., CER-001) to a subject according to an initial “induction” regimen, optionally followed by administering the lipid binding protein-based complex to the subject according to a “consolidation” regimen.

The induction regimen typically comprises administering multiple doses of the lipid binding protein-based complex (e.g., CER-001) to the subject, for example six doses over three days.

The consolidation regimen typically comprises administering one or more doses of a lipid binding protein-based complex (e.g., CER-001) to the subject following the final dose of the induction regimen, for example one or more days after the final dose of the induction regimen. In some embodiments, the first dose of the consolidation regimen is administered on the third day after the final dose of the induction regimen. For example, a dosing regimen can comprise administration of a lipid binding protein-based complex (e.g., CER-001) to a subject according to an induction regimen on days 1, 2, and 3, and administration of the lipid binding protein-based complex to the subject according to a consolidation regimen on day 6. In some embodiments, the consolidation regimen comprises two doses of the lipid binding protein-based complex.

In certain embodiments, the disclosure provides methods of treating a subject having CRS, sepsis or AKI, or a subject at risk of CRS or AKI (e.g., a subject with COVID-19) with a lipid binding protein-based complex (e.g., CER-001) according to a dosage regimen comprising:
2 doses per day on days 1, 2, and 3 (induction regimen) optionally followed by
2 subsequent doses on day 4 or later (consolidation regimen).
   In some embodiments, the regimen comprises:
2 doses per day on days 1, 2, and 3 (induction regimen) followed by
2 doses on day 6 (consolidation regimen).

In certain aspects, a lipid binding protein-based complex (e.g., CER-001) is administered in combination with a standard of care therapy for sepsis such as antibiotic therapy and/or hemodynamic support.

In certain aspects, an antihistamine (e.g., dexchlorpheniramine, hydroxyzine, diphenhydramine, cetirizine, fexofenadine, or loratadine) can be administered before administration of a lipid binding protein-based complex (e.g., CER-001). The antihistamine can reduce the likelihood of allergic reactions.

### 5. BRIEF DESCRIPTION OF THE FIGURES

FIGS. 1A-1D show timelines for four subjects having COVID-19 to whom CER-001 was administered (Example 1). FIG. 1A, Subject 1; FIG. 1B, Subject 2; FIG. 1C, Subject 3; FIG. 1D, Subject 4. DXM=dexamethasone; TCZ=tocilizumab.

FIGS. 2A-2D show apolipoprotein A-I (ApoA-1) levels for Subjects 1-4 of Example 1. FIG. 2A, Subject 1; FIG. 2B, Subject 2; FIG. 2C, Subject 3; FIG. 2D, Subject 4. X-axis shows days measured from first administration of CER-001.

FIGS. 3A-3D show HDL levels for Subjects 1-4 of Example 1. FIG. 3A, Subject 1; FIG. 3B, Subject 2; FIG. 3C, Subject 3; FIG. 3D, Subject 4. X-axis shows days measured from first administration of CER-001.

FIGS. 4A-4D show ferritin levels for Subjects 1-4 of Example 1. FIG. 4A, Subject 1; FIG. 4B, Subject 2; FIG. 4C, Subject 3; FIG. 4D, Subject 4. X-axis shows days measured from first administration of CER-001.

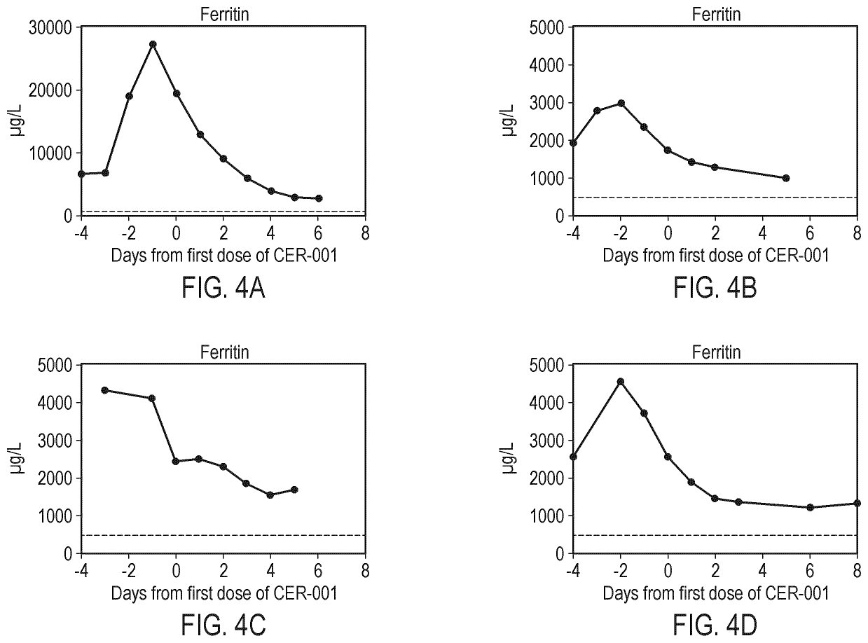

FIGS. 5A-5D show interleukin 8 (IL-8) levels for Subjects 1-4 of Example 1. FIG. 5A, Subject 1; FIG. 5B, Subject 2; FIG. 5C, Subject 3; FIG. 5D, Subject 4. X-axis shows days measured from first administration of CER-001.

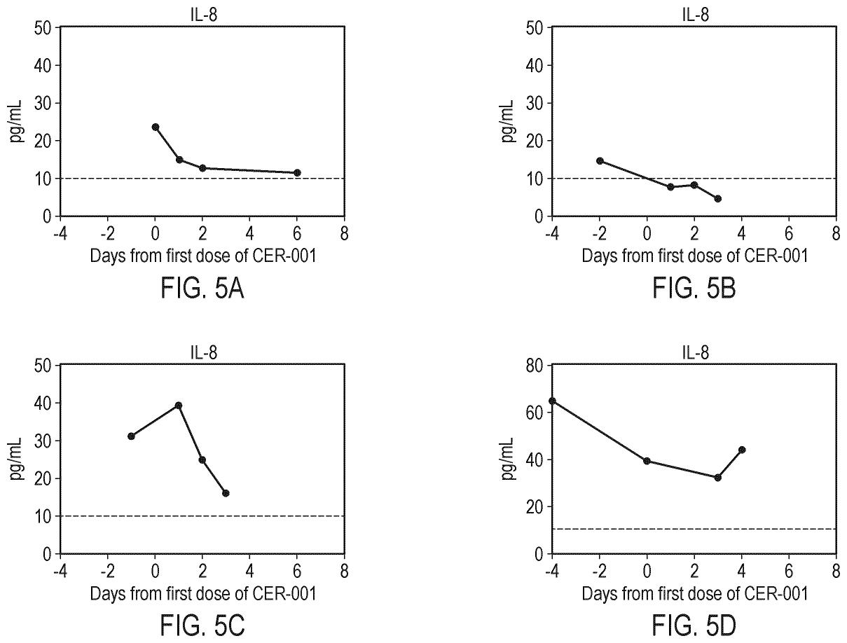

FIGS. 6A-6D show tumor necrosis factor alpha (TNF-α) levels for Subjects 1-4 of Example 1. FIG. 6A, Subject 1; FIG. 6B, Subject 2; FIG. 6C, Subject 3; FIG. 6D, Subject 4. X-axis shows days measured from first administration of CER-001.

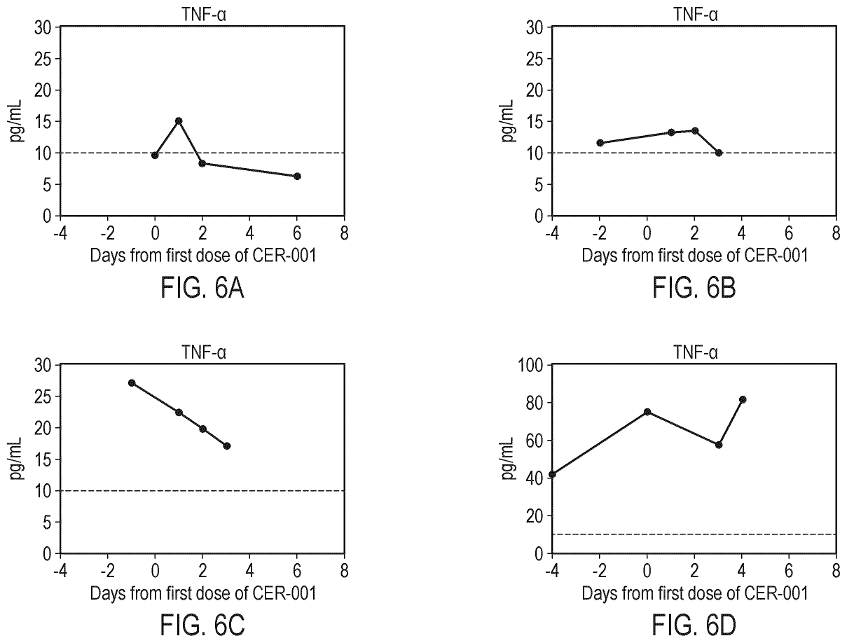

FIGS. 7A-7D show platelet count for Subjects 1-4 of Example 1. FIG. 7A, Subject 1; FIG. 7B, Subject 2; FIG. 7C, Subject 3; FIG. 7D, Subject 4. X-axis shows days measured from first administration of CER-001.

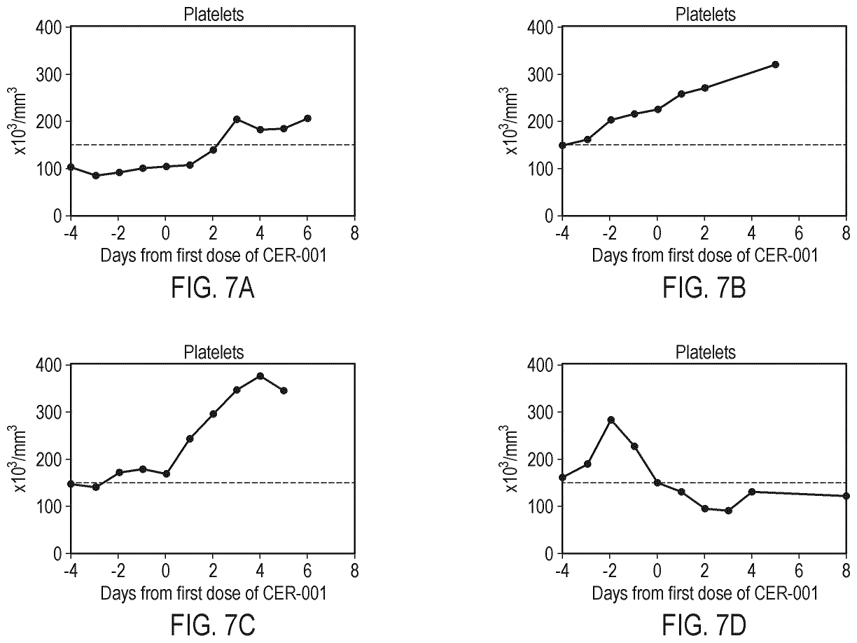

### 6. DETAILED DESCRIPTION

The present disclosure provides methods for treating subjects with acute conditions, for example, an acute condition comprising acute inflammation, with a high dose of a lipid binding protein-based complex (e.g., administered as a formulation comprising a lipid binding protein such as ApoA-I and one or more lipids).

In one aspect, the present disclosure provides methods for treating subjects with an infection, for example a coronavirus infection such as COVID-19, with lipid binding protein-based complexes (e.g., CER-001).

In one aspect, the disclosure provides methods for treating subjects having sepsis using a lipid binding protein-based complex (e.g., CER-001).

In other aspects, the disclosure provides methods for treating subjects with acute kidney injury (AKI) or at risk of AKI (e.g., due to sepsis, viral infection, ischemia/reperfusion, cardiac surgery, or hepatorenal syndrome) using a lipid binding protein-based complex (e.g., CER-001).

In other aspects, the disclosure provides methods of treating a subject with CRS or at risk of CRS, e.g., a subject with CRS secondary to COVID-19 or a subject with CRS caused by immunotherapy.

In some embodiments, the lipid binding protein-based complex is an Apomer, a Cargomer, a HDL based complex, or a HDL mimetic based complex. In specific embodiments, the lipid binding protein-based complex is CER-001.

Exemplary features of lipid binding protein-based complexes that can be used in the methods and compositions of the disclosure are described in Section 6.1. Exemplary subject populations who can be treated by the methods of the disclosure and with the compositions of the disclosure are described in Section 6.2.

In some embodiments, methods of the disclosure comprise administering a lipid binding protein-based complex (e.g., CER-001) to a subject in two phases. First, the lipid binding protein-based complex (e.g., CER-001) is administered in an initial, intense “induction” regimen. The induction regimen is followed by a less intense “consolidation” regimen. Alternatively, a lipid binding protein-based complex (e.g., CER-001) can be administered to a subject in a single phase, for example according to an administration regimen corresponding to the dose and administration frequency of an induction or consolidation regimen described herein.

Induction regimens that can be used in the methods of the disclosure are described in Section 6.3 and consolidation regimens that can be used in the methods of the disclosure are described in Section 6.3.2. The dosing regimens of the disclosure comprise administering a lipid binding protein-based complex (e.g., CER-001) as monotherapy or as part of a combination therapy with one or more medications, for example in combination with a standard of care therapy for sepsis such as antibiotic treatment and/or hemodynamic support. Combination therapies are described in Section 6.4.

### 6.1. Lipid Binding Protein-Based Complexes

### 6.1.1. HDL and HDL Mimetic-Based Complexes

In one aspect, the lipid binding protein-based complexes comprise HDL or HDL mimetic-based complexes. For example, complexes can comprise a lipoprotein complex as described in U.S. Pat. No. 8,206,750, PCT publication WO 2012/109162, PCT publication WO 2015/173633 A2 (e.g., CER-001) or US 2004/0229794 A1, the contents of each of which are incorporated herein by reference in their entireties. The terms “lipoproteins” and “apolipoproteins” are used interchangeably herein, and unless required otherwise by context, the term “lipoprotein” encompasses lipoprotein mimetics. The terms “lipid binding protein” and “lipid binding polypeptide” are also used interchangeably herein, and unless required otherwise by context, the terms do not connote an amino acid sequence of particular length.

Lipoprotein complexes can comprise a protein fraction (e.g., an apolipoprotein fraction) and a lipid fraction (e.g., a phospholipid fraction). The protein fraction includes one or more lipid-binding protein molecules, such as apolipoproteins, peptides, or apolipoprotein peptide analogs or mimetics, for example one or more lipid binding protein molecules described in Section 6.1.2.

The lipid fraction typically includes one or more phospholipids which can be neutral, negatively charged, positively charged, or a combination thereof. Exemplary phospholipids and other amphipathic molecules which can be included in the lipid fraction are described in Section 6.1.3.

In certain embodiments, the lipid fraction contains at least one neutral phospholipid (e.g., a sphingomyelin (SM)) and, optionally, one or more negatively charged phospholipids. In lipoprotein complexes that include both neutral and negatively charged phospholipids, the neutral and negatively charged phospholipids can have fatty acid chains with the same or different number of carbons and the same or different degree of saturation. In some instances, the neutral and negatively charged phospholipids will have the same acyl tail, for example a C16:0, or palmitoyl, acyl chain. In specific embodiments, particularly those in which egg SM is used as the neutral lipid, the weight ratio of the apolipoprotein fraction:lipid fraction ranges from about 1:2.7 to about 1:3 (e.g., 1:2.7).

Any phospholipid that bears at least a partial negative charge at physiological pH can be used as the negatively charged phospholipid. Non-limiting examples include negatively charged forms, e.g., salts, of phosphatidylinositol, a phosphatidylserine, a phosphatidylglycerol and a phosphatidic acid. In a specific embodiment, the negatively charged phospholipid is 1,2-dipalmitoyl-sn-glycero-3-[phospho-rac-(1-glycerol)], or DPPG, a phosphatidylglycerol. Preferred salts include potassium and sodium salts.

In some embodiments, a lipoprotein complex used in the methods of the disclosure is a lipoprotein complex as described in U.S. Pat. No. 8,206,750 or WO 2012/109162 (and its U.S. counterpart, US 2012/0232005), the contents of each of which are incorporated herein in its entirety by reference. In particular embodiments, the protein component of the lipoprotein complex is as described in Section 6.1 and preferably in Section 6.1.1 of WO 2012/109162 (and US 2012/0232005), the lipid component is as described in Section 6.2 of WO 2012/109162 (and US 2012/0232005), which can optionally be complexed together in the amounts described in Section 6.3 of WO 2012/109162 (and US 2012/0232005). The contents of each of these sections are incorporated by reference herein. In certain aspects, a lipoprotein complex of the disclosure is in a population of complexes that is at least 85%, at least 90%, at least 95%, at least 97%, or at least 99% homogeneous, as described in Section 6.4 of WO 2012/109162 (and US 2012/0232005), the contents of which are incorporated by reference herein.

In a specific embodiment, a lipoprotein complex that can be used in the methods of the disclosure comprises 2-4 ApoA-I equivalents, 2 molecules of charged phospholipid, 50-80 molecules of lecithin and 20-50 molecules of SM.

In another specific embodiment, a lipoprotein complex that can be used in the methods of the disclosure comprises 2-4 ApoA-I equivalents, 2 molecules of charged phospholipid, 50 molecules of lecithin and 50 molecules of SM.

In yet another specific embodiment, a lipoprotein complex that can be used in the methods of the disclosure comprises 2-4 ApoA-I equivalents, 2 molecules of charged phospholipid, 80 molecules of lecithin and 20 molecules of SM.

In yet another specific embodiment, a lipoprotein complex that can be used in the methods of the disclosure comprises 2-4 ApoA-I equivalents, 2 molecules of charged phospholipid, 70 molecules of lecithin and 30 molecules of SM.

In yet another specific embodiment, a lipoprotein complex that can be used in the methods of the disclosure comprises 2-4 ApoA-I equivalents, 2 molecules of charged phospholipid, 60 molecules of lecithin and 40 molecules of SM.

In a specific embodiment, a lipoprotein complex that can be used in the methods of the disclosure consists essentially of 2-4 ApoA-I equivalents, 2 molecules of charged phospholipid, 50-80 molecules of lecithin and 20-50 molecules of SM.

In another specific embodiment, a lipoprotein complex that can be used in the methods of the disclosure consists essentially of 2-4 ApoA-I equivalents, 2 molecules of charged phospholipid, 50 molecules of lecithin and 50 molecules of SM.

In yet another specific embodiment, a lipoprotein complex that can be used in the methods of the disclosure consists essentially of 2-4 ApoA-I equivalents, 2 molecules of charged phospholipid, 80 molecules of lecithin and 20 molecules of SM.

In yet another specific embodiment, a lipoprotein complex that can be used in the methods of the disclosure consists essentially of 2-4 ApoA-I equivalents, 2 molecules of charged phospholipid, 70 molecules of lecithin and 30 molecules of SM.

In yet another specific embodiment, a lipoprotein complex that can be used in the methods of the disclosure consists essentially of 2-4 ApoA-I equivalents, 2 molecules of charged phospholipid, 60 molecules of lecithin and 40 molecules of SM.

In a specific embodiment, a lipoprotein complex that can be used in the methods of the disclosure comprises a lipid component that comprises about 90 to 99.8 wt % SM and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, or 0.2-10 wt % total negatively charged phospholipid(s). In another specific embodiment, a lipoprotein complex that can be used in the methods of the disclosure comprises about 90 to 99.8 wt % lecithin and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt % or 0.2-10 wt % total negatively charged phospholipid(s).

In a specific embodiment, a lipoprotein complex that can be used in the methods of the disclosure comprises a lipid component that consists essentially of about 90 to 99.8 wt % SM and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, or 0.2-10 wt % total negatively charged phospholipid(s). In another specific embodiment, a lipoprotein complex that can be used in the methods of the disclosure consists essentially of about 90 to 99.8 wt % lecithin and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt % or 0.2-10 wt % total negatively charged phospholipid(s).

In still another specific embodiment, a lipoprotein complex that can be used in the methods of the disclosure comprises a lipid fraction that comprises about 9.8 to 90 wt % SM, about 9.8 to 90 wt % lecithin and about 0.2-10 wt % negatively charged phospholipid, for example, from about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, to 0.2-10 wt % total negatively charged phospholipid(s).

In still another specific embodiment, a lipoprotein complex that can be used in the methods of the disclosure comprises a lipid fraction that consists essentially of about 9.8 to 90 wt % SM, about 9.8 to 90 wt % lecithin and about 0.2-10 wt % negatively charged phospholipid, for example, from about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, to 0.2-10 wt % total negatively charged phospholipid(s).

In another specific embodiment, a lipoprotein complex that can be used in the methods of the disclosure comprises an ApoA-I apolipoprotein and a lipid fraction, wherein the lipid fraction comprises sphingomyelin and about 3 wt % of a negatively charged phospholipid, wherein the molar ratio of the lipid fraction to the ApoA-I apolipoprotein is about 2:1 to 200:1, and wherein said complex is a small or large discoidal particle containing 2-4 ApoA-I equivalents.

In another specific embodiment, a lipoprotein complex that can be used in the methods of the disclosure comprises an ApoA-I apolipoprotein and a lipid fraction, wherein the lipid fraction consists essentially of sphingomyelin and about 3 wt % of a negatively charged phospholipid, wherein the molar ratio of the lipid fraction to the ApoA-I apolipoprotein is about 2:1 to 200:1, and wherein said complex is a small or large discoidal particle containing 2-4 ApoA-I equivalents.

HDL-based or HDL mimetic-based complexes can include a single type of lipid-binding protein, or mixtures of two or more different lipid-binding proteins, which may be derived from the same or different species. Although not required, the complexes will preferably comprise lipid-binding proteins that are derived from, or correspond in amino acid sequence to, the animal species being treated, in order to avoid inducing an immune response to the therapy. Thus, for treatment of human patients, lipid-binding proteins of human origin are preferably used. The use of peptide mimetic apolipoproteins may also reduce or avoid an immune response.

In some embodiments, the lipid component includes two types of phospholipids: a sphingomyelin (SM) and a negatively charged phospholipid. Exemplary SMs and negatively charged lipids are described in Section 6.1.3.1.

Lipid components including SM can optionally include small quantities of additional lipids. Virtually any type of lipids may be used, including, but not limited to, lysophospholipids, galactocerebroside, gangliosides, cerebrosides, glycerides, triglycerides, and cholesterol and its derivatives.

When included, such optional lipids will typically comprise less than about 15 wt % of the lipid fraction, although in some instances more optional lipids could be included. In some embodiments, the optional lipids comprise less than about 10 wt %, less than about 5 wt %, or less than about 2 wt %. In some embodiments, the lipid fraction does not include optional lipids.

In a specific embodiment, the phospholipid fraction contains egg SM or palmitoyl SM or phytosphingomyelin and DPPG in a weight ratio (SM:negatively charged phospholipid) ranging from 90:10 to 99:1, more preferably ranging from 95:5 to 98:2. In one embodiment, the weight ratio is 97:3.

The molar ratio of the lipid component to the protein component of complexes of the disclosure can vary, and will depend upon, among other factors, the identity(ies) of the apolipoprotein comprising the protein component, the identities and quantities of the lipids comprising the lipid component, and the desired size of the complex. Because the biological activity of apolipoproteins such as ApoA-I are thought to be mediated by the amphipathic helices comprising the apolipoprotein, it is convenient to express the apolipoprotein fraction of the lipid:apolipoprotein molar ratio using ApoA-I protein equivalents. It is generally accepted that ApoA-I contains 6-10 amphipathic helices, depending upon the method used to calculate the helices. Other apolipoproteins can be expressed in terms of ApoA-I equivalents based upon the number of amphipathic helices they contain. For example, ApoA-I_{M}, which typically exists as a disulfide-bridged dimer, can be expressed as 2 ApoA-I equivalents, because each molecule of ApoA-I_{M} contains twice as many amphipathic helices as a molecule of ApoA-I. Conversely, a peptide apolipoprotein that contains a single amphipathic helix can be expressed as a 1/10-1/6 ApoA-I equivalent, because each molecule contains 1/10-1/6 as many amphipathic helices as a molecule of ApoA-I. In general, the lipid:ApoA-1 equivalent molar ratio of the lipoprotein complexes (defined herein as “Ri”) will range from about 105:1 to 110:1. In some embodiments, the Ri is about 108:1. Ratios in weight can be obtained using a MW of approximately 650-800 for phospholipids.

In some embodiments, the molar ratio of lipid:ApoA-I equivalents (“RSM”) ranges from about 80:1 to about 110:1, e.g., about 80:1 to about 100:1. In a specific example, the RSM for complexes can be about 82:1.

In some embodiments, lipoprotein complexes used in the methods of the disclosure are negatively charged complexes which comprise a protein fraction which is preferably mature, full-length ApoA-I, and a lipid fraction comprising a neutral phospholipid, sphingomyelin (SM), and negatively charged phospholipid.

In a specific embodiment, the lipid component contains SM (e.g., egg SM, palmitoyl SM, phytoSM, or a combination thereof) and negatively charged phospholipid (e.g., DPPG) in a weight ratio (SM:negatively charged phospholipid) ranging from 90:10 to 99:1, more preferably ranging from 95:5 to 98:2, e.g., 97:3.

In specific embodiments, the ratio of the protein component to lipid component can range from about 1:2.7 to about 1:3, with 1:2.7 being preferred. This corresponds to molar ratios of ApoA-I protein to lipid ranging from approximately 1:90 to 1:140. In some embodiments, the molar ratio of protein to lipid in the complex is about 1:90 to about 1:120, about 1:100 to about 1:140, or about 1:95 to about 1:125.

In particular embodiments, the complex comprises CER-001, CSL-111, CSL-112, CER-522 or ETC-216. In a preferred embodiment, the complex is CER-001.

CER-001 as used in the literature and in the Examples below refers to a complex described in Example 4 of WO 2012/109162. WO 2012/109162 refers to CER-001 as a complex having a 1:2.7 lipoprotein weight:total phospholipid weight ratio with a SM:DPPG weight:weight ratio of 97:3. Example 4 of WO 2012/109162 also describes a method of its manufacture.

When used in the context of a method and/or CER-001 dosing regimen of the disclosure, CER-001 refers to a lipoprotein complex whose individual constituents can vary from CER-001 as described in Example 4 of WO 2012/109162 by up to 20%. In certain embodiments, the constituents of the lipoprotein complex vary from CER-001 as described in Example 4 of WO 2012/109162 by up to 10%. Preferably, the constituents of the lipoprotein complex are those described in Example 4 of WO 2012/109162 (plus/minus acceptable manufacturing tolerance variations). The SM in CER-001 can be natural or synthetic. In some embodiments, the SM is a natural SM, for example a natural SM described in WO 2012/109162, e.g., chicken egg SM. In some embodiments, the SM is a synthetic SM, for example a synthetic SM described in WO 2012/109162, e.g., synthetic palmitoylsphingomyelin, for example as described in WO 2012/109162. Methods for synthesizing palmitoylsphingomyelin are known in the art, for example as described in WO 2014/140787. The lipoprotein in CER-001, apolipoprotein A-1 (ApoA-1), preferably has an amino acid sequence corresponding to amino acids 25 to 267 of SEQ ID NO:2 (previously published as SEQ ID NO:1 of WO 2012/109162). ApoA-I can be purified by animal sources (and in particular from human sources) or produced recombinantly. In preferred embodiments, the ApoA-I in CER-001 is recombinant ApoA-I. CER-001 used in a dosing regimen of the disclosure is preferably highly homogeneous, for example at least 80%, at least 85%, at least 90%, at least 95%, at least 97%, at least 98%, or at least 99% homogeneous, as reflected by a single peak in gel permeation chromatography. See, e.g., Section 6.4 of WO 2012/109162.

In particular embodiments, the ApoA-I in CER-001 is recombinant ApoA-I produced by a mammalian host cell. The host cell can be from any mammalian cell line. The polynucleotides encoding the ApoA-I can be codon optimized for expression in recombinant host cells. Preferred host cells are mammalian host cells, including, but not limited, Chinese hamster ovary cells (e.g. CHO-K1; ATCC No. CCL 61; CHO-S (GIBCO Life Technologies Inc., Rockville, MD, Catalog #11619012)), VERO cells, BHK (ATCC No. CRL 1632), BHK 570 (ATCC No. CRL 10314), HeLa cells, COS-1 (ATCC No. CRL 1650), COS-7 (ATCC No. CRL 1651), MDCK cells, 293 cells (ATCC No. CRL 1573; Graham et al., J. Gen. Virol. 36:59-72, 1977), 3T3 cells, myeloma cells (especially murine), PC12 cells and W138 cells. In certain embodiments, the mammalian cells, such as CHO-S cells (Invitrogen™, Carlsbad CA), are adapted for growth in serum-free medium. Additional suitable cell lines are known in the art and available from public depositories such as the American Type Culture Collection, Manassas, Va.

In a preferred embodiment, the recombinant ApoA-I is produced by a CHO cell. Recombinant ApoA-I expressed by a mammalian host cell, such as a CHO cell, may undergo post-translational processing (e.g., glycosylation, etc.). The resulting recombinant ApoA-I can have one or more structural features (e.g., glycosylation pattern) that are different from ApoA-I purified from human plasma.

For recombinant expression of ApoA-I, the polynucleotides encoding ApoA-I are operably linked to one or more control sequences, e.g., a promoter or terminator, that regulate the expression of ApoA-I in the host cell of interest. The control sequence(s) can be native or foreign to the ApoA-I-encoding sequence, and also native or foreign to the host cell in which the ApoA-I is expressed. Control sequences include, but are not limited to, promoters, ribosome binding sites, leaders, polyadenylation sequences, propeptide sequences, signal peptide sequences, and transcription terminators. In some embodiments, the control sequences include a promoter, ribosome binding site, and transcriptional and translational stop signals. The control sequences can also include one or more linkers for the purpose of introducing specific restriction sites facilitating ligation of the control sequences with the coding region of the nucleotide sequence encoding ApoA-I.

The promoters driving the recombinant expression of ApoA-I can be constitutive promoters, regulated promoters, or inducible promoters. Appropriate promoter sequences can be obtained from genes encoding extracellular or intracellular polypeptides which are either endogenous or heterologous to the host cell. Methods for the isolation, identification and manipulation of promoters of varying strengths are available in or readily adapted from the art. See e.g., Nevoigt et al. (2006) Appl. Environ. Microbiol. 72:5266-5273, the disclosure of which is herein incorporated by reference in its entirety.

One or more of the control sequences can be derived from viral sources. For example, in certain aspects, promoters are derived from polyoma or adenovirus major late promoter. In other aspects, the promoter is derived from Simian Virus 40 (SV40), which can be obtained as a fragment that also contains the SV40 viral origin of replication (Fiers et al., 1978, Nature, 273:113-120), or from cytomegalovirus, e.g., simian cytomegalovirus immediate early promoter. (See U.S. Pat. No. 4,956,288). Other suitable promoters include those from metallothionein genes (See U.S. Pat. Nos. 4,579,821 and 4,601,978).

Also provided herein are recombinant ApoA-I expression vectors. A recombinant expression vector can be any vector, e.g., a plasmid or a virus, that can be manipulated by recombinant DNA techniques to facilitate expression of a heterologous ApoA-I in a recombinant host cell. The expression vector can be integrated into the chromosome of the recombinant host cell and comprises one or more heterologous genes operably linked to one or more control sequences useful for production of ApoA-I. In other embodiments, the expression vector is an extrachromosomal replicative DNA molecule, e.g., a linear or closed circular plasmid, that is found either in low copy number (e.g., from about 1 to about 10 copies per genome equivalent) or in high copy number (e.g., more than about 10 copies per genome equivalent). In various embodiments, the expression vector includes a selectable marker, such as a gene that confers antibiotic resistance (e.g., ampicillin, kanamycin, chloramphenicol or tetracycline resistance) to the recombinant host organism that comprises the vector. In particular aspects, the DNA constructs, vectors and polynucleotides are suitable for expression of ApoA-I in mammalian cells. Vectors for expression of ApoA-I in mammalian cells can include an origin of replication, a promoter and any necessary ribosome binding sites, RNA splice sites, polyadenylation site, and transcriptional terminator sequences that are compatible with the host cell systems. In some aspects, an origin of replication is heterologous to the host cell, e.g., is of viral origin (e.g., SV40, Polyoma, Adeno, VSV, BPV). In other aspects, an origin of replication is provided by the host cell chromosomal replication mechanism.

Methods, reagents and tools for introducing foreign DNA into mammalian host cells are known in the art and include, but are not limited to, calcium phosphate-mediated transfection (Wigler et al., 1978, Cell 14:725; Corsaro et al., 1981, Somatic Cell Genetics 7:603; Graham et al., 1973, Virology 52:456), electroporation (Neumann et al., 1982, EMBO J. 1:841-5), DEAE-dextran mediated transfection (Ausubel et al. (eds.), Short Protocols in Molecular Biology, 3^{rd} Edition (John Wiley & Sons 1995)), and liposome-mediated transfection (Hawley-Nelson et al., 1993, Focus 15:73; Ciccarone et al., 1993, Focus 15:80).

For high-yield production, stable expression of ApoA-I is preferred. For example, following the introduction of foreign DNA into the host cells, the host cells may be allowed to grow for 1-2 days in an enriched media, and then are switched to a selective media. Rather than using expression vectors that contain viral origins of replication, host cells can be transformed with vector comprising a nucleotide sequence comprising the ApoA-l-coding sequence controlled by appropriate expression control elements and a selectable marker. The selectable marker in the vector confers resistance to the selection and allows cells to stably integrate the vector into their chromosomes and grow to form foci which in turn can be cloned and expanded into cell lines. A number of selection systems may be used, including but not limited to the herpes simplex virus thymidine kinase (Wigler et al., 1977, Cell 11: 223), hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) genes can be employed in tk-, hgprt- or aprt-cells, respectively. Also, antimetabolite resistance can be used as the basis of selection by using, for example, dhfr, which confers resistance to methotrexate (Wigler et al., 1980, Natl. Acad. Sci. USA 77: 3567; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072; neo, which confers resistance to the aminoglycoside G-418 (Colberre-Garapin et al., 1981, J. Mol. Biol. 150: 1); and/or hyg, which confers resistance to hygromycin (Santerre et al., 1984, Gene 30: 147).

Stable, high yield expression can also be achieved using retroviral vectors that integrate into the host cell genome (see, e.g., U.S. Patent Publications No. 2008/0286779 and 2004/0235173). Alternatively, stable, high yield expression of ApoA-I can be achieved by gene activation methods, which entail activating expression of and amplifying an endogenous ApoA-I gene in genomic DNA of a mammalian cell of choice, for example as described in WO 1994/012650. Increasing the copy number of an ApoA-I gene (containing an ApoA-I coding sequence and one or more control elements) can facilitate the high yield expression of ApoA-I. Preferably, the mammalian host cell in which ApoA-I is expressed has an ApoA-I gene copy index of at least 2, at least 3, at least 4, or at least 5. In specific embodiments, the mammalian host cell in which ApoA-I is expressed has an ApoA-I gene copy index of at least 6, at least 7, at least 8, at least 9, or at least 10.

In certain embodiments, the mammalian cells are adapted to produce ApoA-I in quantities of at least 0.5 g/L, at least 1 g/L, at least 1.5 g/L, at least 2 g/L, at least 2.5 g/L, at least 3 g/L, at least 3.5 g/L, and optionally up to 4 g/L, up to 4.5 g/L, up to 5 g/L, up to 5.5 g/L, or up to 6 g/L. The mammalian host cells are preferably capable of producing at least about 0.5, 1, 2, or 3 g/L ApoA-I in culture and/or up to about 20 g/L ApoA-I in culture, e.g., up to 4, 5, 6, 7, 8, 9, 10, 12, or 15 g/L ApoA-I in culture.

In certain embodiments, the mammalian cells are adapted for growth in serum-free medium. In these embodiments, the ApoA-I is secreted from the cells. In other embodiments, the ApoA-I is not secreted from the cells.

The mammalian host cells provided herein can be used to produce ApoA-I. Generally, the methods comprise culturing a mammalian host cell as described herein under conditions in which ApoA-I is expressed. Furthermore, the methods can comprise recovering and, optionally, purifying mature ApoA-I from the supernatant of the mammalian cell culture.

The culture conditions, including the culture medium, temperature, pH, can be suited to the mammalian host cell being cultured and the mode of culture chosen (shake flask, bioreactor, roller bottle, etc. . . . ). Mammalian cells can be grown in large scale batch culture, in continuous or semi-continuous culture.

Also provided herein is a mammalian cell culture comprising a plurality of a ApoA-1-producing mammalian host cells described herein. In some embodiments, the mammalian cell culture comprises at least 0.5 g/L, at least 1 g/L, at least 1.5 g/L, at least 2 g/L, at least 2.5 g/L, at least 3 g/L, at least 3.5 g/L, and optionally up to 4 g/L, up to 4.5 g/L, up to 5 g/L, up to 5.5 g/L, or up to 6 g/L of ApoA-I. The culture can be of any scale, ranging from about 150 mL to about 500 mL, 1 L, 10 L, 15 L, 50 L, 100 L, 200 L, 250 L, 300 L, 350 L, 400 L, 500 L, 750 L, 1000 L, 1500 L, 2000 L, 2500 L, 3000 L, 5000 L, 7500 L, 10000 L, 15000 L, 20000 L, 25000 L, 50000 L or more. In some instances, the culture is a large scale culture, such as 15 L, 20 L, 25 L, 30 L, 50 L, 100 L, 200 L, 300 L, 500 L, 1000 L, 5000 L, 10000 L, 15000 L, 20000 L, 25000 L, up to 50000 L or more.

The mammalian host cells of the present disclosure can be grown in culture. Thus, the present disclosure further provides a mammalian cell culture, comprising a plurality of mammalian host cells as described above. The cell culture can include one or more of the following features: (a) the culture (which is optionally a large scale batch culture of at least 10 liters, at least 20 liters, at least 30 liters, at least 50 liters, at least 100 liters, 300 L, 500 L, 1000 L, 5000 L, 10,000 L, 15,000 L, 20,000 L, 25,000 L, up to 50,000 L or a continuous culture of at least 10 liters, at least 20 liters, at least 30 liters, at least 50 liters, at least 100 liters, 300 L, 500 L, 1000 L, 5000 L, or up to 10,000 L) comprises at least about 0.5, 1.0, 1.5, 2.0, 2.5, 3.0, 3.5, 4.0 g/L or more of mature ApoA-I protein comprising or consisting of an amino sequence corresponding to amino acids 25 to 267 of SEQ ID NO:2; (b) at least 75%, at least 80%, at least 85%, at least 90%, at least 95%, at least 98%, or at least 99% of the protein in the culture medium is an ApoA-I protein lacking a signal sequence; (c) at least 75%, at least 80%, at least 85%, at least 90%, at least 95%, at least 98%, or at least 99% of the protein in the culture medium is a mature ApoA-I protein lacking a signal sequence and a propeptide sequence; and (d) at least 75%, at least 80%, at least 85%, at least 90%, at least 95% of the mature ApoA-I is not truncated, oxidized, or deamidated.

CSL-111 is a reconstituted human ApoA-I purified from plasma complexed with soybean phosphatidylcholine (SBPC) (Tardif et al., 2007, JAMA 297:1675-1682).

CSL-112 is a formulation of ApoA-I purified from plasma and reconstituted to form HDL suitable for intravenous infusion (Diditchenko et al., 2013, DOI 10.1161/ATVBAHA.113.301981).

ETC-216 (also known as MDCO-216) is a lipid-depleted form of HDL containing recombinant ApoA-I_{Milano}. See Nicholls et al., 2011, Expert Opin Biol Ther. 11(3):387-94. doi: 10.1517/14712598.2011.557061.

In another embodiment, a complex that can be used in the methods of the disclosure is CER-522. CER-522 is a lipoprotein complex comprising a combination of three phospholipids and a 22 amino acid peptide, CT80522:

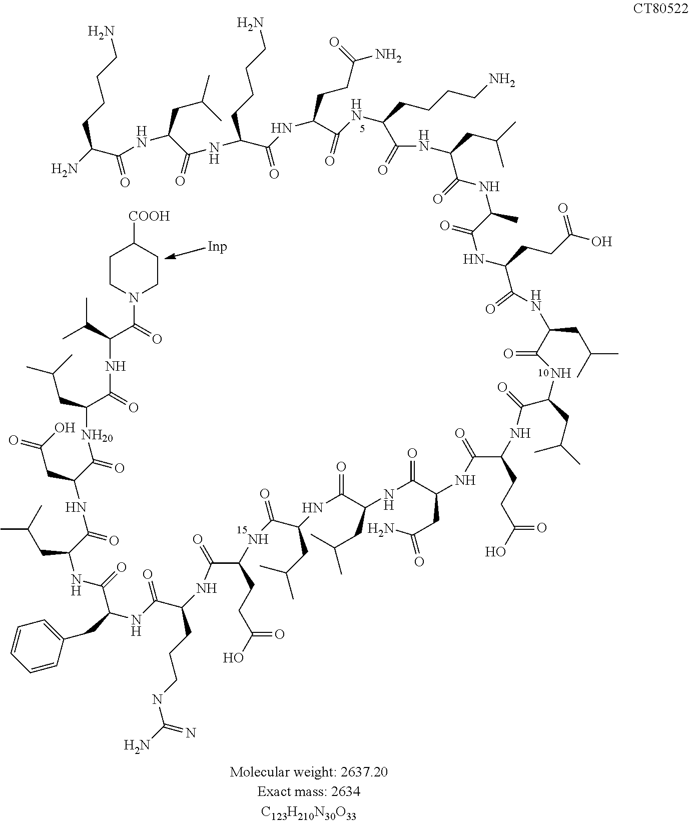 [SMILES: CC(C)C[C@H](NC(=O)[C@H](CCCCN)NC(=O)[C@H](CCC(N)=O)NC(=O)[C@H](CCCCN)NC(=O)[C@H](CC(C)C)NC(=O)[C@@H](N)CCCCN)C(=O)N[C@@H](C)C(=O)N[C@@H](CCC(=O)O)C(=O)N[C@@H](CC(C)C)C(=O)N[C@@H](CC(C)C)C(=O)N[C@@H](CCC(=O)O)C(=O)N[C@@H](CC(N)=O)C(=O)N[C@@H](CC(C)C)C(=O)N[C@@H](CC(C)C)C(=O)N[C@@H](CCC(=O)O)C(=O)N[C@@H](CCCNC(=N)N)C(=O)N[C@@H](Cc1ccccc1)C(=O)N[C@@H](CC(C)C)C(=O)N[C@@H](CC(=O)O)C(=O)N[C@@H](CC(C)C)C(=O)N[C@H](C(=O)N1CCC(C(=O)O)CC1)C(C)C]

The phospholipid component of CER-522 consists of egg sphingomyelin,1,2-dipalmitoyl-sn-glycero-3-phosphocholine (Dipalmitoylphosphatidylcholine, DPPC) and 1,2-dipalmitoyl-sn-glycero-3-[phospho-rac-(1-glycerol)] (Dipalmitoylphosphatidyl-glycerol, DPPG) in a 48.5:48.5:3 weight ratio. The ratio of peptide to total phospholipids in the CER-522 complex is 1:2.5 (w/w).

In some embodiments, the lipoprotein complex is delipidated HDL. Most HDL in plasma is cholesterol-rich. The lipids in HDL can be depleted, for example partially and/or selectively depleted, e.g., to reduce its cholesterol content. In some embodiments, the delipidated HDL can resemble small a, prep-1, and other prep forms of HDL. A process for selective depletion of HDL is described in Sacks et al., 2009, J Lipid Res. 50(5): 894-907.

In certain embodiments, a lipoprotein complex comprises a bioactive agent delivery particle as described in US 2004/0229794.

A bioactive agent delivery particle can comprise a lipid binding polypeptide (e.g., an apolipoprotein as described previously in this Section or in Section 6.1.2), a lipid bilayer (e.g., comprising one or more phospholipids as described previously in this Section or in Section 6.1.3.1), and a bioactive agent (e.g., an anti-cancer agent), wherein the interior of the lipid bilayer comprises a hydrophobic region, and wherein the bioactive agent is associated with the hydrophobic region of the lipid bilayer. In some embodiments, a bioactive agent delivery particle as described in US 2004/0229794.

In some embodiments, a bioactive agent delivery particle does not comprise a hydrophilic core.

In some embodiments, a bioactive agent delivery particle is disc shaped (e.g., having a diameter from about 7 to about 29 nm).

Bioactive agent delivery particles include bilayer-forming lipids, for example phospholipids (e.g., as described previously in this Section or in Section 6.1.3.1). In some embodiments, a bioactive agent delivery particle includes both bilayer-forming and non-bilayer-forming lipids. In some embodiments, the lipid bilayer of a bioactive agent delivery particle includes phospholipids. In one embodiment, the phospholipids incorporated into a delivery particle include dimyristoylphosphatidylcholine (DMPC) and dimyristoylphosphatidylglycerol (DMPG). In one embodiment, the lipid bilayer includes DMPC and DMPG in a 7:3 molar ratio.

In some embodiments, the lipid binding polypeptide is an apolipoprotein (e.g., as described previously in this Section or in Section 6.1.2). The predominant interaction between lipid binding polypeptides, e.g., apolipoprotein molecules, and the lipid bilayer is generally a hydrophobic interaction between residues on a hydrophobic face of an amphipathic structure, e.g., an α-helix of the lipid binding polypeptide and fatty acyl chains of lipids on an exterior surface at the perimeter of the particle. Bioactive agent delivery particles may include exchangeable and/or non-exchangeable apolipoproteins. In one embodiment, the lipid binding polypeptide is ApoA-I.

In some embodiments, bioactive agent delivery particles include lipid binding polypeptide molecules, e.g., apolipoprotein molecules, that have been modified to increase stability of the particle. In one embodiment, the modification includes introduction of cysteine residues to form intramolecular and/or intermolecular disulfide bonds.

In another embodiment, bioactive agent delivery particles include a chimeric lipid binding polypeptide molecule, e.g., a chimeric apolipoprotein molecule, with one or more bound functional moieties, for example one or more targeting moieties and/or one or more moieties having a desired biological activity, e.g., antimicrobial activity, which may augment or work in synergy with the activity of a bioactive agent incorporated into the delivery particle.

### 6.1.2. Lipid Binding Protein Molecules

Lipid binding protein molecules that can be used in the complexes described herein include apolipoproteins such as those described in Section 6.1.2.1 and apolipoprotein mimetic peptides such as those described in Section 6.1.2.2. In some embodiments, the complex comprises a mixture of lipid binding protein molecules. In some embodiments, the complex comprises a mixture of one or more lipid binding protein molecules and one or more apolipoprotein mimetic peptides.

In some embodiments, the complex comprises 1 to 8 ApoA-I equivalents (e.g., 1, 2, 3, 4, 5, 6, 7, 8, 1 to 7, 1 to 6, 1 to 5, 1 to 4, 1 to 3, 1 to 2, 2 to 8, 2 to 6, 2 to 4, 4 to 6, or 4 to 8 ApoA-I equivalents). Lipid binding proteins can be expressed in terms of ApoA-I equivalents based upon the number of amphipathic helices they contain. For example, ApoA-I_{M}, which typically exists as a disulfide-bridged dimer, can be expressed as 2 ApoA-I equivalents, because each molecule of ApoA-I_{M} contains twice as many amphipathic helices as a molecule of ApoA-I. Conversely, a peptide mimetic that contains a single amphipathic helix can be expressed as a 1/10-1/6 ApoA-I equivalent, because each molecule contains 1/10-1/6 as many amphipathic helices as a molecule of ApoA-I.

### 6.1.2.1. Apolipoproteins

Suitable apolipoproteins that can be included in the lipid binding protein-based complexes include apolipoproteins ApoA-I, ApoA-II, ApoA-IV, ApoA-V, ApoB, ApoC-I, ApoC-II, ApoC-III, ApoD, ApoE, ApoJ, ApoH, and any combination of two or more of the foregoing. Polymorphic forms, isoforms, variants and mutants as well as truncated forms of the foregoing apolipoproteins, the most common of which are Apolipoprotein A-Imilano (ApoA-IM), Apolipoprotein A-Iparis (ApoA-IP), and Apolipoprotein A-Izaragoza (ApoA-IZ), can also be used. Apolipoproteins mutants containing cysteine residues are also known, and can also be used (see, e.g., U.S. Publication No. 2003/0181372). The apolipoproteins may be in the form of monomers or dimers, which may be homodimers or heterodimers. For example, homo- and heterodimers (where feasible) of ApoA-I (Duverger et al., 1996, Arterioscler. Thromb. Vasc. Biol. 16(12):1424-29), ApoA-IM (Franceschini et al., 1985, J. Biol. Chem. 260:1632-35), ApoA-IP (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), ApoE (McLean et al., 1983, J. Biol. Chem. 258(14):8993-9000), ApoJ and ApoH may be used.

The apolipoproteins can be modified in their primary sequence to render them less susceptible to oxidations, for example, as described in U.S. Publication Nos. 2008/0234192 and 2013/0137628, and U.S. Pat. Nos. 8,143,224 and 8,541,236. The apolipoproteins can include residues corresponding to elements that facilitate their isolation, such as His tags, or other elements designed for other purposes. Preferably, the apolipoprotein in the complex is soluble in a biological fluid (e.g., lymph, cerebrospinal fluid, vitreous humor, aqueous humor, blood, or a blood fraction (e.g., serum or plasma).

In some embodiments, the complex comprises covalently bound lipid-binding protein monomers, e.g., dimeric apolipoprotein A-Imilano, which is a mutated form of ApoA-I containing a cysteine. The cysteine allows the formation of a disulfide bridge which can lead to the formation of homodimers or heterodimers (e.g., ApoA-I Milano-ApoA-II).

In some embodiments, the apolipoprotein molecules comprise ApoA-I, ApoA-II, ApoA-IV, ApoA-V, ApoB, ApoC-I, ApoC-II, ApoC-Ill, ApoD, ApoE, ApoJ, or ApoH molecules or a combination thereof.

In some embodiments, the apolipoprotein molecules comprise or consist of ApoA-I molecules. In some embodiments, said ApoA-I molecules are human ApoA-I molecules. In some embodiments, said ApoA-I molecules are recombinant. In some embodiments, the ApoA-I molecules are not ApoA-Imilano.

In some embodiments, the ApoA-I molecules are Apolipoprotein A-Imilano (ApoA-IM), Apolipoprotein A-Iparis (ApoA-IP), or Apolipoprotein A-Izaragoza (ApoA-IZ) molecules.

Apolipoproteins can be purified from animal sources (and in particular from human sources) or produced recombinantly as is well-known in the art, see, e.g., Chung et al., 1980, J. Lipid Res. 21(3):284-91; Cheung et al., 1987, J. Lipid Res. 28(8):913-29. See also U.S. Pat. Nos. 5,059,528, 5,128,318, 6,617,134; U.S. Publication Nos. 2002/0156007, 2004/0067873, 2004/0077541, and 2004/0266660; and PCT Publications Nos. WO 2008/104890 and WO 2007/023476. Other methods of purification are also possible, for example as described in PCT Publication No. WO 2012/109162, the disclosure of which is incorporated herein by reference in its entirety. ApoA-I molecules can be produced recombinantly, for example in CHO cells, as described elsewhere herein.

The apolipoprotein can be in prepro-form, pro-form, or mature form. For example, a complex can comprise ApoA-I (e.g., human ApoA-I) in which the ApoA-I is preproApoA-1, proApoA-I, or mature ApoA-I. In some embodiments, the complex comprises ApoA-I that has at least 90% sequence identity to SEQ ID NO:1:

(SEQ ID NO: 1)
PPQSPWDRVKDLATVYVDVLKDSGRDYVSQFEGSALGKQLNLKLLDNWDS
VTSTFSKLREQLGPVTQEFWDNLEKETEGLRQEMSKDLEEVKAKVQPYLD
DFQKKWQEEMELYRQKVEPLRAELQEGARQKLHELQEKLSPLGEEMRDRA
RAHVDALRTHLAPYSDELRQRLAARLEALKENGGARLAEYHAKATEHLST
LSEKAKPALEDLRQGLLPVLESFKVSFLSALEEYTKKLNTQ

In other embodiments, the complex comprises ApoA-I that has at least 95% sequence identity to SEQ ID NO:1. In other embodiments, the complex comprises ApoA-I that has at least 98% sequence identity to SEQ ID NO:1. In other embodiments, the complex comprises ApoA-I that has at least 99% sequence identity to SEQ ID NO:1. In other embodiments, the complex comprises ApoA-I that has 100% sequence identity to SEQ ID NO:1.

In some embodiments, the complex comprises ApoA-I that has at least 90% sequence identity to amino acids 25 to 267 of SEQ ID NO:2:

(SEQ ID NO: 2)
MKAAVLTLAVLFLTGSQARHFWQQDEPPQSPWDRVKDLATVYVDVLKDSG
RDYVSQFEGSALGKQLNLKLLDNWDSVTSTFSKLREQLGPVTQEFWDNLE
KETEGLRQEMSKDLEEVKAKVQPYLDDFQKKWQEEMELYRQKVEPLRAEL
QEGARQKLHELQEKLSPLGEEMRDRARAHVDALRTHLAPYSDELRQRLAA
RLEALKENGGARLAEYHAKATEHLSTLSEKAKPALEDLRQGLLPVLESFK
VSFLSALEEYTKKLNTQ.

In other embodiments, the complex comprises ApoA-I that has at least 95% sequence identity to amino acids 25 to 267 of SEQ ID NO:2. In other embodiments, the complex comprises ApoA-I that has at least 98% sequence identity to amino acids 25 to 267 of SEQ ID NO:2. In other embodiments, the complex comprises ApoA-I that has at least 99% sequence identity to amino acids 25 to 267 of SEQ ID NO:2. In other embodiments, the complex comprises ApoA-I that has 100% sequence identity to amino acids 25 to 267 of SEQ ID NO:2.

In some embodiments, the complex comprises 1 to 8 apolipoprotein molecules (e.g., 1 to 6, 1 to 4, 1 to 2, 2 to 8, 2 to 6, 2 to 4, 4 to 8, 4 to 6, or 6 to 8 apolipoprotein molecules). In some embodiments, the complex comprises 1 apolipoprotein molecule. In some embodiments, the complex comprises 2 apolipoprotein molecules. In some embodiments, the complex comprises 3 apolipoprotein molecules. In some embodiments, the complex comprises 4 apolipoprotein molecules. In some embodiments, the complex comprises 5 apolipoprotein molecules. In some embodiments, the complex comprises 6 apolipoprotein molecules. In some embodiments, the complex comprises 7 apolipoprotein molecules. In some embodiments, the complex comprises 8 apolipoprotein molecules.

The apolipoprotein molecule(s) can comprise a chimeric apolipoprotein comprising an apolipoprotein and one or more attached functional moieties, such as for example, one or more CRN-001 complex(es), one or more targeting moieties, a moiety having a desired biological activity, an affinity tag to assist with purification, and/or a reporter molecule for characterization or localization studies. An attached moiety with biological activity may have an activity that is capable of augmenting and/or synergizing with the biological activity of a compound incorporated into a complex of the disclosure. For example, a moiety with biological activity may have antimicrobial (for example, antifungal, antibacterial, anti-protozoal, bacteriostatic, fungistatic, or antiviral) activity. In one embodiment, an attached functional moiety of a chimeric apolipoprotein is not in contact with hydrophobic surfaces of the complex. In another embodiment, an attached functional moiety is in contact with hydrophobic surfaces of the complex. In some embodiments, a functional moiety of a chimeric apolipoprotein may be intrinsic to a natural protein. In some embodiments, a chimeric apolipoprotein includes a ligand or sequence recognized by or capable of interaction with a cell surface receptor or other cell surface moiety.

In one embodiment, a chimeric apolipoprotein includes a targeting moiety that is not intrinsic to the native apolipoprotein, such as for example, S. cerevisiae α-mating factor peptide, folic acid, transferrin, or lactoferrin. In another embodiment, a chimeric apolipoprotein includes a moiety with a desired biological activity that augments and/or synergizes with the activity of a compound incorporated into a complex of the disclosure. In one embodiment, a chimeric apolipoprotein may include a functional moiety intrinsic to an apolipoprotein. One example of an apolipoprotein intrinsic functional moiety is the intrinsic targeting moiety formed approximately by amino acids 130-150 of human ApoE, which comprises the receptor binding region recognized by members of the low density lipoprotein receptor family. Other examples of apolipoprotein intrinsic functional moieties include the region of ApoB-100 that interacts with the low density lipoprotein receptor and the region of ApoA-I that interacts with scavenger receptor type B 1. In other embodiments, a functional moiety may be added synthetically or recombinantly to produce a chimeric apolipoprotein. Another example is an apolipoprotein with the prepro or pro sequence from another preproapolipoprotein (e.g., prepro sequence from preproapoA-II substituted for the prepro sequence of preproapoA-I). Another example is an apolipoprotein for which some of the amphipathic sequence segments have been substituted by other amphipathic sequence segments from another apolipoprotein.

As used herein, “chimeric” refers to two or more molecules that are capable of existing separately and are joined together to form a single molecule having the desired functionality of all of its constituent molecules. The constituent molecules of a chimeric molecule may be joined synthetically by chemical conjugation or, where the constituent molecules are all polypeptides or analogs thereof, polynucleotides encoding the polypeptides may be fused together recombinantly such that a single continuous polypeptide is expressed. Such a chimeric molecule is termed a fusion protein. A “fusion protein” is a chimeric molecule in which the constituent molecules are all polypeptides and are attached (fused) to each other such that the chimeric molecule forms a continuous single chain. The various constituents can be directly attached to each other or can be coupled through one or more linkers. One or more segments of various constituents can be, for example, inserted in the sequence of an apolipoprotein, or, as another example, can be added N-terminal or C-terminal to the sequence of an apolipoprotein. For example, a fusion protein can comprise an antibody light chain, an antibody fragment, a heavy-chain antibody, or a single-domain antibody.

In some embodiments, a chimeric apolipoprotein is prepared by chemically conjugating the apolipoprotein and the functional moiety to be attached. Means of chemically conjugating molecules are well known to those of skill in the art. Such means will vary according to the structure of the moiety to be attached, but will be readily ascertainable to those of skill in the art. Polypeptides typically contain a variety of functional groups, e.g., carboxylic acid (—COOH), free amino (—NH2), or sulfhydryl (—SH) groups, that are available for reaction with a suitable functional group on the functional moiety or on a linker to bind the moiety thereto. A functional moiety may be attached at the N-terminus, the C-terminus, or to a functional group on an interior residue (i.e., a residue at a position intermediate between the N- and C-termini) of an apolipoprotein molecule. Alternatively, the apolipoprotein and/or the moiety to be tagged can be derivatized to expose or attach additional reactive functional groups.

In some embodiments, fusion proteins that include a polypeptide functional moiety are synthesized using recombinant expression systems. Typically, this involves creating a nucleic acid (e.g., DNA) sequence that encodes the apolipoprotein and the functional moiety such that the two polypeptides will be in frame when expressed, placing the DNA under the control of a promoter, expressing the protein in a host cell, and isolating the expressed protein.

A nucleic acid encoding a chimeric apolipoprotein can be incorporated into a recombinant expression vector in a form suitable for expression in a host cell. As used herein, an “expression vector” is a nucleic acid which, when introduced into an appropriate host cell, can be transcribed and translated into a polypeptide. The vector may also include regulatory sequences such as promoters, enhancers, or other expression control elements (e.g., polyadenylation signals). Such regulatory sequences are known to those skilled in the art (see, e.g., Goeddel, 1990, Gene Expression Technology: Meth. Enzymol. 185, Academic Press, San Diego, Calif.; Berger and Kimmel, Guide to Molecular Cloning Techniques, Methods in Enzymology 152 Academic Press, Inc., San Diego, Calif.; Sambrook et al., 1989, Molecular Cloning—A Laboratory Manual (2^{nd} ed.) Vol. 1-3, Cold Spring Harbor Laboratory, Cold Spring Harbor Press, NY, etc.).

In some embodiments, an apolipoprotein has been modified such that when the apolipoprotein is incorporated into a complex of the disclosure, the modification will increase stability of the complex, confer targeting ability or increase capacity. In one embodiment, the modification includes introduction of cysteine residues into apolipoprotein molecules to permit formation of intramolecular or intermolecular disulfide bonds, e.g., by site-directed mutagenesis. In another embodiment, a chemical crosslinking agent is used to form intermolecular links between apolipoprotein molecules to enhance stability of the complex. Intermolecular crosslinking prevents or reduces dissociation of apolipoprotein molecules from the complex and/or prevents displacement by endogenous apolipoprotein molecules within an individual to whom the complexes are administered. In other embodiments, an apolipoprotein is modified either by chemical derivatization of one or more amino acid residues or by site directed mutagenesis, to confer targeting ability to or recognition by a cell surface receptor.

Complexes can be targeted to a specific cell surface receptor by engineering receptor recognition properties into an apolipoprotein. For example, complexes may be targeted to a particular cell type known to harbor a particular type of infectious agent, for example by modifying the apolipoprotein to render it capable of interacting with a receptor on the surface of the cell type being targeted. For example, complexes may be targeted to macrophages by altering the apolipoprotein to confer recognition by the macrophage endocytic class A scavenger receptor (SR-A). SR-A binding ability can be conferred to a complex by modifying the apolipoprotein by site directed mutagenesis to replace one or more positively charged amino acids with a neutral or negatively charged amino acid. SR-A recognition can also be conferred by preparing a chimeric apolipoprotein that includes an N- or C-terminal extension having a ligand recognized by SR-A or an amino acid sequence with a high concentration of negatively charged residues. Complexes comprising apoplipoproteins can also interact with apolipoprotein receptors such as, but not limited to, ABCA1 receptors, ABCG1 receptors, Megalin, Cubulin and HDL receptors such as SR-B1.

### 6.1.2.2. Apolipoprotein Mimetics

Peptides, peptide analogs, and agonists that mimic the activity of an apolipoprotein (collectively referred to herein as “apolipoprotein peptide mimetics”) can also be used in the complexes described herein, either alone, in combination with one or more other lipid binding proteins. Non-limiting examples of peptides and peptide analogs that correspond to apolipoproteins, as well as agonists that mimic the activity of ApoA-I, ApoA-I_{M}, ApoA-II, ApoA-IV, and ApoE, that are suitable for inclusion in the complexes and compositions described herein are disclosed in U.S. Pat. Nos. 6,004,925, 6,037,323 and 6,046,166 (issued to Dasseux et al.), U.S. Pat. No. 5,840,688 (issued to Tso), U.S. Pat. No. 6,743,778 (issued to Kohno), U.S. Publication Nos. 2004/0266671, 2004/0254120, 2003/0171277 and 2003/0045460 (to Fogelman), U.S. Publication No. 2006/0069030 (to Bachovchin), U.S. Publication No. 2003/0087819 (to Bielicki), U.S. Publication No. 2009/0081293 (to Murase et al.), and PCT Publication No. WO/2010/093918 (to Dasseux et al.), the disclosures of which are incorporated herein by reference in their entireties. These peptides and peptide analogues can be composed of L-amino acid or D-amino acids or mixture of L- and D-amino acids. They may also include one or more non-peptide or amide linkages, such as one or more well-known peptide/amide isosteres. Such apolipoprotein peptide mimetic can be synthesized or manufactured using any technique for peptide synthesis known in the art, including, e.g., the techniques described in U.S. Pat. Nos. 6,004,925, 6,037,323 and 6,046,166.

In some embodiments, the lipid binding protein molecules comprise apolipoprotein peptide mimetic molecules and optionally one or more apolipoprotein molecules such as those described above.

In some embodiments, the apolipoprotein peptide mimetic molecules comprise an ApoA-I peptide mimetic, ApoA-II peptide mimetic, ApoA-IV peptide mimetic, or ApoE peptide mimetic or a combination thereof.

### 6.1.3. Amphipathic Molecules

An amphipathic molecule is a molecule that possesses both hydrophobic (apolar) and hydrophilic (polar) elements. Amphipathic molecules that can be used in complexes described herein include lipids (e.g., as described in Section 6.1.3.1), detergents (e.g., as described in Section 6.1.3.2), fatty acids (e.g., as described in Section 6.1.3.3), and apolar molecules and sterols covalently attached to polar molecules such as, but not limited to, sugars or nucleic acids (e.g., as described in Section 6.1.3.4).

The complexes can include a single class of amphipathic molecule (e.g., a single species of phospholipids or a mixture of phospholipids) or can contain a combination of classes of amphipathic molecules (e.g., phospholipids and detergents). The complex can contain one species of amphipathic molecules or a combination of amphipathic molecules configured to facilitate solubilization of the lipid binding protein molecule(s).

In some embodiments, the amphipathic molecules included in comprise a phospholipid, a detergent, a fatty acid, an apolar moiety or sterol covalently attached to a sugar, or a combination thereof (e.g., selected from the types of amphipathic molecules discussed above).

In some embodiments, the amphipathic molecules comprise or consist of phospholipid molecules. In some embodiments, the phospholipid molecules comprise negatively charged phospholipids, neutral phospholipids, positively charged phospholipids or a combination thereof. In some embodiments, the phospholipid molecules contribute a net charge of 1-3 per apolipoprotein molecule in the complex. In some embodiments, the net charge is a negative net charge. In some embodiments, the net charge is a positive net charge. In some embodiments, the phospholipid molecules consist of a combination of negatively charged and neutral phospholipids. In some embodiments, the molar ratio of negatively charge phospholipid to neutral phospholipid ranges from 1:1 to 1:3. In some embodiments, the molar ratio of negatively charged phospholipid to neutral phospholipid is about 1:1 or about 1:2.

In some embodiments, the amphipathic molecules comprise neutral phospholipids and negatively charged phospholipids in a weight ratio of 95:5 to 99:1.

### 6.1.3.1. Lipids

Lipid binding protein-based complexes can include one or more lipids. In various embodiments, one or more lipids can be saturated and/or unsaturated, natural and/or synthetic, charged or not charged, zwitterionic or not. In some embodiments, the lipid molecules (e.g., phospholipid molecules) can together contribute a net charge of 1-3 (e.g., 1-3, 1-2, 2-3, 1, 2, or 3) per lipid binding protein molecule in the complex. In some embodiments, the net charge is negative. In other embodiments, the net charge is positive.

In some embodiments, the lipid comprises a phospholipid. Phospholipids can have two acyl chains that are the same or different (for example, chains having a different number of carbon atoms, a different degree of saturation between the acyl chains, different branching of the acyl chains, or a combination thereof). The lipid can also be modified to contain a fluorescent probe (e.g., as described at avantilipids.com/product-category/products/fluorescent-lipids/). Preferably, the lipid comprises at least one phospholipid.

Phospholipids can have unsaturated or saturated acyl chains ranging from about 6 to about 24 carbon atoms (e.g., 6-20, 6-16, 6-12, 12-24, 12-20, 12-16, 16-24, 16-20, or 20-24). In some embodiments, a phospholipid used in a complex of the disclosure has one or two acyl chains of 12, 14, 16, 18, 20, 22, or 24 carbons (e.g., two acyl chains of the same length or two acyl chains of different length).

Non-limiting examples of acyl chains present in commonly occurring fatty acids that can be included in phospholipids are provided in Table 1, below:

TABLE 1

| Length:Number of Unsaturations | Common Name |
|---|---|
| 14:0 | myristic acid |
| 16:0 | palmitic acid |
| 18:0 | stearic acid |
| 18:1 cisΔ⁹ | oleic acid |
| 18:2 cisΔ^{9, 12} | linoleic acid |
| 18:3 cisΔ^{9, 12, 15} | linonenic acid |
| 20:4 cisΔ^{5, 8, 11, 14} | arachidonic acid |
| 20:5 cisΔ^{5, 8, 11, 14, 17} | eicosapentaenoic acid |
| | (an omega-3 fatty acid) |

Lipids that can be present in the complexes of the disclosure include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, palmitoylsphingomyelin, dipalmitoylsphingomyelin, egg sphingomyelin, milk sphingomyelin, phytosphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives. Synthetic lipids, such as synthetic palmitoylsphingomyelin or N-palmitoyl-4-hydroxysphinganine-1-phosphocholine (a form of phytosphingomyelin) can be used to minimize lipid oxidation.

In some embodiments, a lipid binding protein-based complex includes two types of phospholipids: a neutral lipid, e.g., lecithin and/or sphingomyelin (abbreviated SM), and a charged phospholipid (e.g., a negatively charged phospholipid). A “neutral” phospholipid has a net charge of about zero at physiological pH. In many embodiments, neutral phospholipids are zwitterions, although other types of net neutral phospholipids are known and can be used. In some embodiments, the molar ratio of the charged phospholipid (e.g., negatively charged phospholipid) to neutral phospholipid ranges from 1:1 to 1:3, for example, about 1:1, about 1:2, or about 1:3.

The neutral phospholipid can comprise, for example, one or both of the lecithin and/or SM, and can optionally include other neutral phospholipids. In some embodiments, the neutral phospholipid comprises lecithin, but not SM. In other embodiments, the neutral phospholipid comprises SM, but not lecithin. In still other embodiments, the neutral phospholipid comprises both lecithin and SM. All of these specific exemplary embodiments can include neutral phospholipids in addition to the lecithin and/or SM, but in many embodiments do not include such additional neutral phospholipids.

As used herein, the expression “SM” includes sphingomyelins derived or obtained from natural sources, as well as analogs and derivatives of naturally occurring SMs that are impervious to hydrolysis by LCAT, as is naturally occurring SM. SM is a phospholipid very similar in structure to lecithin, but, unlike lecithin, it does not have a glycerol backbone, and hence does not have ester linkages attaching the acyl chains. Rather, SM has a ceramide backbone, with amide linkages connecting the acyl chains. SM can be obtained, for example, from milk, egg or brain. SM analogues or derivatives can also be used. Non-limiting examples of useful SM analogues and derivatives include, but are not limited to, palmitoylsphingomyelin, N-palmitoyl-4-hydroxysphinganine-1-phosphocholine (a form of phytosphingomyelin), palmitoylsphingomyelin, stearoylsphingomyelin, D-erythro-N-16:0-sphingomyelin and its dihydro isomer, D-erythro-N-16:0-dihydro-sphingomyelin. Synthetic SM such as synthetic palmitoylsphingomyelin or N-palmitoyl-4-hydroxysphinganine-1-phosphocholine (phytosphingomyelin) can be used in order to produce more homogeneous complexes and with fewer contaminants and/or oxidation products than sphingolipids of animal origin. Methods for synthesizing SM are described in U.S. Publication No. 2016/0075634.

Sphingomyelins isolated from natural sources can be artificially enriched in one particular saturated or unsaturated acyl chain. For example, milk sphingomyelin (Avanti Phospholipid, Alabaster, Ala.) is characterized by long saturated acyl chains (i.e., acyl chains having 20 or more carbon atoms). In contrast, egg sphingomyelin is characterized by short saturated acyl chains (i.e., acyl chains having fewer than 20 carbon atoms). For example, whereas only about 20% of milk sphingomyelin comprises C16:0 (16 carbon, saturated) acyl chains, about 80% of egg sphingomyelin comprises C16:0 acyl chains. Using solvent extraction, the composition of milk sphingomyelin can be enriched to have an acyl chain composition comparable to that of egg sphingomyelin, or vice versa.

The SM can be semi-synthetic such that it has particular acyl chains. For example, milk sphingomyelin can be first purified from milk, then one particular acyl chain, e.g., the C16:0 acyl chain, can be cleaved and replaced by another acyl chain. The SM can also be entirely synthesized, by e.g., large-scale synthesis. See, e.g., Dong et al., U.S. Pat. No. 5,220,043, entitled Synthesis of D-erythro-sphingomyelins, issued Jun. 15, 1993; Weis, 1999, Chem. Phys. Lipids 102 (1-2):3-12. SM can be fully synthetic, e.g., as described in U.S. Publication No. 2014/0275590.

The lengths and saturation levels of the acyl chains comprising a semi-synthetic or a synthetic SM can be selectively varied. The acyl chains can be saturated or unsaturated, and can contain from about 6 to about 24 carbon atoms. Each chain can contain the same number of carbon atoms or, alternatively each chain can contain different numbers of carbon atoms. In some embodiments, the semi-synthetic or synthetic SM comprises mixed acyl chains such that one chain is saturated and one chain is unsaturated. In such mixed acyl chain SMs, the chain lengths can be the same or different. In other embodiments, the acyl chains of the semi-synthetic or synthetic SM are either both saturated or both unsaturated. Again, the chains can contain the same or different numbers of carbon atoms. In some embodiments, both acyl chains comprising the semi-synthetic or synthetic SM are identical. In a specific embodiment, the chains correspond to the acyl chains of a naturally-occurring fatty acid, such as for example oleic, palmitic or stearic acid. In another embodiment, SM with saturated or unsaturated functionalized chains is used. In another specific embodiment, both acyl chains are saturated and contain from 6 to 24 carbon atoms. Non-limiting examples of acyl chains present in commonly occurring fatty acids that can be included in semi-synthetic and synthetic SMs are provided in Table 1, above.

In some embodiments, the SM is palmitoyl SM, such as synthetic palmitoyl SM, which has C16:0 acyl chains, or is egg SM, which includes as a principal component palmitoyl SM.

In a specific embodiment, functionalized SM, such as phytosphingomyelin, is used.

Lecithin can be derived or isolated from natural sources, or it can be obtained synthetically. Examples of suitable lecithins isolated from natural sources include, but are not limited to, egg phosphatidylcholine and soybean phosphatidylcholine. Additional non-limiting examples of suitable lecithins include, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoy1-2-palmitoylphosphatidylcholine, 1-palmitoy1-2-oleoylphosphatidylcholine, 1-oleoy1-2-palmitylphosphatidylcholine, dioleoylphosphatidylcholine and the ether derivatives or analogs thereof.

Lecithins derived or isolated from natural sources can be enriched to include specified acyl chains. In embodiments employing semi-synthetic or synthetic lecithins, the identity(ies) of the acyl chains can be selectively varied, as discussed above in connection with SM. In some embodiments of the complexes described herein, both acyl chains on the lecithin are identical. In some embodiments of complexes that include both SM and lecithin, the acyl chains of the SM and lecithin are all identical. In a specific embodiment, the acyl chains correspond to the acyl chains of myristitic, palmitic, oleic or stearic acid.

The complexes of the disclosure can include one or more negatively charged phospholipids (e.g., alone or in combination with one or more neutral phospholipids). As used herein, “negatively charged phospholipids” are phospholipids that have a net negative charge at physiological pH. The negatively charged phospholipid can comprise a single type of negatively charged phospholipid, or a mixture of two or more different, negatively charged, phospholipids. In some embodiments, the charged phospholipids are negatively charged glycerophospholipids. Specific examples of suitable negatively charged phospholipids include, but are not limited to, a 1,2-dipalmitoyl-sn-glycero-3-[phospho-rac-(1-glycerol)], a phosphatidylglycerol, a phospatidylinositol, a phosphatidylserine, a phosphatidic acid, and salts thereof (e.g., sodium salts or potassium salts). In some embodiments, the negatively charged phospholipid comprises one or more of phosphatidylinositol, phosphatidylserine, phosphatidylglycerol and/or phosphatidic acid. In a specific embodiment, the negatively charged phospholipid comprises or consists of a salt of a phosphatidylglycerol or a salt of a phosphatidylinositol. In another specific embodiment, the negatively charged phospholipid comprises or consists of 1,2-dipalmitoyl-sn-glycero-3-[phospho-rac-(1-glycerol)], or DPPG, or a salt thereof.

The negatively charged phospholipids can be obtained from natural sources or prepared by chemical synthesis. In embodiments employing synthetic negatively charged phospholipids, the identities of the acyl chains can be selectively varied, as discussed above in connection with SM. In some embodiments of the complexes of the disclosure, both acyl chains on the negatively charged phospholipids are identical. In some embodiments, the acyl chains all types of phospholipids included in a complex of the disclosure are all identical. In a specific embodiment, the complex comprises negatively charged phospholipid(s), and/or SM all having C16:0 or C16:1 acyl chains. In a specific embodiment the fatty acid moiety of the SM is predominantly C16:1 palmitoyl. In one specific embodiment, the acyl chains of the charged phospholipid(s), lecithin and/or SM correspond to the acyl chain of palmitic acid. In yet another specific embodiment, the acyl chains of the charged phospholipid(s), lecithin and/or SM correspond to the acyl chain of oleic acid.

Examples of positively charged phospholipids that can be included in the complexes of the disclosure include N1-[2-((1S)-1-[(3-aminopropyl)amino]-4-[di(3-amino-propyl)amino]butylcarboxamido)ethyl]-3,4-di[oleyloxy]-benzamide, 1,2-di-O-octadecenyl-3-trimethylammonium propane, 1,2-dimyristoleoyl-sn-glycero-3-ethylphosphocholine, 1-palmitoyl-2-oleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1,2-dilauroyl-sn-glycero-3-ethylphosphocholine, 1,2-dioleoyl-3-dimethylammonium-propane1,2-dimyristoyl-3-dimethylammonium-propane, 1,2-dipalmitoyl-3-dimethylammonium-propane, N-(4-carboxybenzyl)-N,N-dimethyl-2,3-bis(oleoyloxy)propan-1-aminium, 1,2-dioleoyl-3-trimethylammonium-propane, 1,2-dioleoyl-3-trimethylammonium-propane, 1,2-stearoyl-3-trimethylammonium-propane, 1,2-dipalmitoyl-3-trimethylammonium-propane, 1,2-dimyristoyl-3-trimethylammonium-propane, N-[1-(2,3-dimyristyloxy)propyl]-N, N-dimethyl-N-(2-hydroxyethyl) ammonium bromide, N,N,N-trimethyl-2-bis[(1-oxo-9-octadecenyl)oxy]-(Z,Z)-1propanaminium methyl sulfate, and salts thereof (e.g., chloride or bromide salts).

The lipids used are preferably at least 95% pure, and/or have reduced levels of oxidative agents (such as but not limited to peroxides). Lipids obtained from natural sources preferably have fewer polyunsaturated fatty acid moieties and/or fatty acid moieties that are not susceptible to oxidation. The level of oxidation in a sample can be determined using an iodometric method, which provides a peroxide value, expressed in milli-equivalent number of isolated iodines per kg of sample, abbreviated meq O/kg. See, e.g., Gray, 1978, Measurement of Lipid Oxidation: A Review, Journal of the American Oil Chemists Society 55:539-545; Heaton, F.W. and Ur, Improved Iodometric Methods for the Determination of Lipid Peroxides, 1958, Journal of the Science of Food and Agriculture 9:781-786. Preferably, the level of oxidation, or peroxide level, is low, e.g., less than 5 meq O/kg, less than 4 meq O/kg, less than 3 meq O/kg, or less than 2 meq O/kg.

Complexes can in some embodiments include small quantities of additional lipids. Virtually any type of lipids can be used, including, but not limited to, lysophospholipids, galactocerebroside, gangliosides, cerebrosides, glycerides, triglycerides, and sterols and sterol derivatives (e.g., a plant sterol, an animal sterol, such as cholesterol, or a sterol derivative, such as a cholesterol derivative). For example, a complex of the disclosure can contain cholesterol or a cholesterol derivative, e.g., a cholesterol ester. The cholesterol derivative can also be a substituted cholesterol or a substituted cholesterol ester. The complexes of the disclosure can also contain an oxidized sterol such as, but not limited to, oxidized cholesterol or an oxidized sterol derivative (such as, but not limited to, an oxidized cholesterol ester). In some embodiments, the complexes do not include cholesterol and/or its derivatives (such as a cholesterol ester or an oxidized cholesterol ester).

### 6.1.3.2. Detergents

The complexes can contain one or more detergents. The detergent can be zwitterionic, nonionic, cationic, anionic, or a combination thereof. Exemplary zwitterionic detergents include 3-[(3-Cholamidopropyl)dimethylammonio]-1-propanesulfonate (CHAPS), 3-[(3-Cholamidopropyl)dimethylammonio]-2-hydroxy-1-propanesulfonate (CHAPSO), and N,N-dimethyldodecylamine N-oxide (LDAO). Exemplary nonionic detergents include D-(+)-trehalose 6-monooleate, N-octanoyl-N-methylglucamine, N-nonanoyl-N-methylglucamine, N-decanoyl-N-methylglucamine, 1-(7Z-hexadecenoyl)-rac-glycerol, 1-(8Z-hexadecenoyl)-rac-glycerol, 1-(8Z-heptadecenoyl)-rac-glycerol, 1-(9Z-hexadecenoyl)-rac-glycerol, 1-decanoyl-rac-glycerol. Exemplary cationic detergents include (S)—O-methyl-serine dodecylamide hydrochloride, dodecylammonium chloride, decyltrimethylammonium bromide, and cetyltrimethylammonium sulfate. Exemplary anionic detergents include cholesteryl hemisuccinate, cholate, alkyl sulfates, and alkyl sulfonates.

### 6.1.3.3. Fatty Acids

The complexes can contain one or more fatty acids. The one or more fatty acids can include short-chain fatty acids having aliphatic tails of five or fewer carbons (e.g. butyric acid, isobutyric acid, valeric acid, or isovaleric acid), medium-chain fatty acids having aliphatic tails of 6 to 12 carbons (e.g., caproic acid, caprylic acid, capric acid, or lauric acid), long-chain fatty acids having aliphatic tails of 13 to 21 carbons (e.g., myristic acid, palmitic acid, stearic acid, or arachidic acid), very long chain fatty acids having aliphatic tails of 22 or more carbons (e.g., behenic acid, lignoceric acid, or cerotic acid), or a combination thereof. The one or more fatty acids can be saturated (e.g., caprylic acid, capric acid, lauric acid, myristic acid, palmitic acid, stearic acid, arachidic acid, behenic acid, lignoceric acid, or cerotic acid), unsaturated (e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, or docosahexaenoic acid) or a combination thereof. Unsaturated fatty acids can be cis or trans fatty acids. In some embodiments, unsaturated fatty acids used in the complexes of the disclosure are cis fatty acids.

### 6.1.3.4. Apolar Molecules and Sterols Attached to a Sugar

The complexes can contain one or more amphipathic molecules that comprise an apolar molecule or moiety (e.g., a hydrocarbon chain, an acyl or diacyl chain) or a sterol (e.g., cholesterol) attached to a sugar (e.g., a monosaccharide such as glucose or galactose, or a disaccharide such as maltose or trehalose). The sugar can be a modified sugar or a substituted sugar. Exemplary amphipathic molecules comprising an apolar molecule attached to a sugar include dodecan-2-yloxy-β-D-maltoside, tridecan-3-yloxy-β-D-maltoside, tridecan-2-yloxy-β-D-maltoside, n-dodecyl-β-D-maltoside (DDM), n-octyl-β-D-glucoside, n-nonyl-β-D-glucoside, n-decyl-β-D-maltoside, n-dodecyl-β-D-maltopyranoside, 4-n-Dodecyl-α,α-trehalose, 6-n-dodecyl-α,α-trehalose, and 3-n-dodecyl-α,α-trehalose.

In some embodiments, the apolar moiety is an acyl or a diacyl chain.

In some embodiments, the sugar is a modified sugar or a substituted sugar.

### 6.1.4. Formulations

Lipid binding protein-based complexes can be formulated for the intended route of administration, for example according to techniques known in the art (e.g., as described in Allen et al., eds., 2012, Remington: The Science and Practice of Pharmacy, 22^{nd} Edition, Pharmaceutical Press, London, UK).

CER-001 intended for administration by infusion can be formulated in a phosphate buffer with sucrose and mannitol excipients, for example as described in WO 2012/109162.

### 6.2. Subject Populations

Subjects who can be treated according to the methods described herein are preferably mammals, most preferably human.

In some aspects, the subject has an acute condition comprising acute inflammation.

In some aspects, the subject has an infection, for example a coronavirus infection such as COVID-19.

In some aspects, the subject can be a subject in need of therapy for sepsis and/or AKI.

In some embodiments, the subject has sepsis (e.g., associated with a gram-negative bacterial infection). The sepsis can in some embodiments be caused by an intra-abdominal cavity infection or be urosepsis. Sepsis is a risk factor for AKI. Thus, in some embodiments, the subject can be at risk for AKI, for example due to sepsis. In some embodiments, the subject has sepsis associated with a gram negative bacterial infection. In other embodiments, the subject has sepsis associated with a gram positive bacterial infection.

In some embodiments, the subject has a total SOFA score of 2 to 24 before treatment with a lipid binding protein-based complex, e.g., a total score of 2 to 5, 2 to 10, 5 to 10, 5 to 15, 10 to 15, 10 to 20, or 15 to 24. The SOFA scoring system is described in Vincent et al. 1996, Intensive Care Med, 22:707-710, and uses a 0 to 4 scale to assess function of six different body systems (respiratory, cardiovascular, hepatic, coagulation, renal, and neurological). The sum of the individual scores for the six different body systems provides a subject's total SOFA score. In some embodiments, the subject has a total SOFA score of 2 to 5 before treatment with a lipid binding protein-based complex. In other embodiments, the subject has a total SOFA score of 2 to 10 before treatment with a lipid binding protein-based complex. In other embodiments, the subject has a total SOFA score of 5 to 15 before treatment with a lipid binding protein-based complex. In other embodiments, the subject has a total SOFA score of 8 to 13 before treatment with a lipid binding protein-based complex.

In some embodiments, the subject has an endotoxin activity level as measured by the Endotoxin Activity Assay (EAA™) (Spectral Medical) of >0.6 prior to administration of the lipid binding protein-based complex (see, Marshall et al., 2004, J Infect Dis. 190(3):527-34).

In some embodiments, the subject has AKI or is at risk of AKI. For example, a the AKI can be sepsis-related AKI, ischemia/reperfusion AKI, CSA-AKI, or hepatorenal syndrome (HSA) AKI. In some embodiments, the AKI is sepsis-related AKI. In other embodiments, the AKI is ischemia/reperfusion AKI. In other embodiments, the AKI is CSA AKI. In other embodiments, the AKI is HRS AKI. Subjects at risk of HRS include subjects having liver disease (e.g., chronic liver disease or acute liver disease). In some embodiments, the subject has chronic liver disease. In some embodiments, the subject has acute liver disease. In some embodiments, the subject has alcoholic liver disease. HRS has historically been classified as type 1 HRS, where renal function rapidly deteriorates over days to weeks, and type 2 HRS, where deterioration occurs over months. Accordingly, in some embodiments, a subject treated according to a dosage regimen of the disclosure has type 1 HRS. In other embodiments, a subject treated according to a dosage regiment of the disclosure has type 2 HRS. Newer criteria for diagnosis and classification of HRS have been developed, for example the ICA diagnostic criteria of HRS acute kidney injury (AKI). See, e.g., Amin et al., 2019, Seminars in Nephrology 39(1):17-30. Accordingly, in some embodiments, a subject having HRS meets the ICA diagnostic criteria of HRS AKI.

In some aspects, the subject can be any subject having CRS or at risk of CRS, and/or any subject in need of reduction in serum levels of one or more inflammatory markers such as IL-6. In some embodiments, the subject has CRS. In some embodiments, the subject has CRS secondary to an infection, for example a viral infection such as an infection with COVID-19 or influenza. In some embodiments, the subject has CRS secondary to a COVID-19 infection. In other embodiments, the subject has CRS caused by immunotherapy, for example antibody or chimeric antigen receptor (CAR) T cell therapy. In yet other embodiments, the subject is at risk of CRS, for example due to an infection such as COVID-19 or influenza. In other embodiments, the subject is at risk of CRS due to immunotherapy.

In another aspect, the subject is a subject in need of a reduction in serum levels of one or more inflammatory markers, for example a subject with elevated levels of the one or more inflammatory markers compared to normal levels. Exemplary inflammatory cytokines include interleukin 6 (IL-6), C-reactive protein, D-dimer, ferritin, interleukin 8 (IL-8), granulocyte-macrophage colony stimulating factor (GM-CSF), monocyte chemoattractant protein (MCP) 1, and tumor necrosis factor α (TNFα). In some embodiments, the one or more cytokines comprise IL-6. In some embodiments, the one or more cytokines comprise a combination of the foregoing, for example, 2, 3, 4, 5, 6, 7, or all 8 of interleukin 6 (IL-6), C-reactive protein, D-dimer, ferritin, interleukin 8 (IL-8), granulocyte-macrophage colony stimulating factor (GM-CSF), monocyte chemoattractant protein (MCP) 1, and tumor necrosis factor α (TNFα).

### 6.3. Dosing Regimens

The methods of the disclosure typically entail multiple administrations of a lipid binding protein-based complex (e.g., CER-001), e.g., two to 10 individual doses, although in some embodiments, a single dose may be used. In some embodiments, an administration regimen can include two or more, three or more, or four or more doses of a lipid binding protein-based complex (e.g., CER-001), e.g., five, six, seven, eight, nine, ten, eleven, twelve, or more than twelve doses. In some embodiments, an administration regimen comprises or consists of a single dose. In some embodiments, an administration regimen comprises or consists of two individual doses. In some embodiments, an administration regimen comprises or consists of three individual doses. In some embodiments, an administration regimen comprises or consists of four individual doses.

In some embodiments, the lipid binding protein-based complex is administered according to an induction and, optionally, a consolidation regimen as described in Sections 6.3.1 and 6.3.2, respectively. In some embodiments, the lipid binding protein-based complex can be administered in a single phase, e.g., according to an administration regimen described in this Section. In some embodiments, the subject is not treated with the lipid binding protein-based complex according to a maintenance regimen, e.g., a regimen comprising long-term (e.g., one month or longer) administration of the lipid binding protein-based complex.

The lipid binding protein-based complex (e.g., CER-001) administration regimens of the disclosure can last up to one week, one week, or more than one week (e.g., two weeks).

For example, a lipid binding protein-based complex (e.g., CER-001) administration regimen can comprise administering:
five doses of CER-001 over one week;
six doses of CER-001 over one week;
seven doses of CER-001 over one week;
ten doses of CER-001 over two weeks;
twelve doses of CER-001 over two weeks;
fourteen doses of CER-001 over two weeks.

In an embodiment, the methods of the disclosure (e.g., methods for treating CRS or a subject at risk of CRS) comprise administering seven doses of CER-001 over one week, e.g., on days 1, 2, 3, 4, 5, 6, and 7.

In some embodiments, of the methods of the disclosure, a plurality of doses of a lipid binding protein-based complex (e.g., CER-001) are administered no more than one day apart. For example, in some embodiments two or more individual doses are administered approximately 12 hours apart. In some embodiments, two individual doses are administered approximately 12 hours apart. In other embodiments, three individual doses are administered approximately 12 hours apart. In other embodiments, two individual doses are administered approximately 12 hours apart and a third individual dose is administered approximately one day later. In other embodiments, three individual doses are administered approximately 12 hours apart and a fourth individual dose is administered approximately one day later.

In some embodiments of the methods of the disclosure, a lipid binding protein-based complex (e.g., CER-001) is administered to a subject (e.g., over a period of 0.5 to 1 hour) at hours 0 and 12, for example at a dose of 10 mg/kg or 15 mg/kg. In some embodiments of the methods of the disclosure, a lipid binding protein-based complex (e.g., CER-001) is administered to a subject (e.g., over a period of 0.5 to 1 hour) at hours 0 and 12, 24, and 48, for example at a dose of 10 mg/kg or 15 mg/kg.

In some embodiments of the methods of the disclosure, a lipid binding protein-based complex (e.g., CER-001) is administered daily, e.g., daily for at least 5 days, at least 6 days, at least 7 days, or more than 7 days (e.g., daily for up to one week or daily for up to two weeks). In other embodiments, a lipid binding protein-based complex (e.g., CER-001) is administered less frequently, e.g., every other day, two times per week, three times per week, or once a week.

In practice, an administration window can be provided, for example, to accommodate slight variations to a multi-dosing per week dosing schedule. For example, a window of ±2 days or ±1 day around the dosage date can be used.

A lipid binding protein-based complex (e.g., CER-001) can be administered in the methods of the disclosure for a pre-determined period of time, e.g., for one week. Alternatively, administration of a lipid binding protein-based complex (e.g., CER-001) can be continued until one or more symptoms of the acute indication (e.g., CRS) are reduced or continued until the serum levels of one or more inflammatory markers are reduced, for example reduced to a normal level or reduced relative to a baseline value for the subject, e.g., a baseline value measured prior to the start of lipid binding protein-based complex (e.g., CER-001) therapy. Reference or “normal” levels of various inflammatory markers are known in the art. For example, the Mayo Clinic Laboratories test catalog (mayocliniclabs.com/test-catalog) provides the following reference values: IL-6: ≤1.8 μg/ml; C-reactive protein: ≤8.0 mg/ml; D-dimer: ≤500 ng/mL Fibrinogen Equivalent Units (FEU); ferritin: 24-336 mcg/L (males), 11-307 mcg/L (females); IL-8<57.8 μg/mL; TNF-α<5.6 μg/mL.

When administering a lipid binding protein-based complex (e.g., CER-001) to a subject who has CRS due to immunotherapy or is at risk of CRS due to immunotherapy, a lipid binding protein-based complex (e.g., CER-001) can be administered before the immunotherapy begins, concurrently with the immunotherapy, after the immunotherapy ends, or a combination thereof. For example, a lipid binding protein-based complex (e.g., CER-001) can be administered before the immunotherapy and currently with the immunotherapy, concurrently with the immunotherapy and after the immunotherapy, or before the immunotherapy, concurrently with the immunotherapy and after the immunotherapy. Concurrent administration is not limited to administration of the lipid binding protein-based complex (e.g., CER-001) and the immunotherapy at the exact same time, and encompasses administration of one agent while a course of therapy with the other is ongoing.

The methods of the disclosure (e.g., methods for treating an acute condition described herein) typically comprise administering a high dose of a lipid binding protein-based complex (e.g., CER-001). The high dose can be the aggregate of multiple individual doses (e.g., two, three, four, five, six, seven, eight, nine or 10 individual doses), for example administered over one or multiple days (e.g., a period of one day, a period of two days, a period of three days, four days, five days, six days, seven days, eight days, nine days, 10 days, eleven days, 12 days, 13 days, 14 days or 15 days). The individual doses of a high dose are in some embodiments administered daily, twice daily, or two to three days apart.

In some embodiments, the high dose is an amount effective to increase the subject's HDL and/or ApoA-I blood levels and/or improve the subject's vascular endothelial function, e.g., measured by circulating vascular cell adhesion molecule 1 (VCAM-1) and/or intercellular adhesion molecule 1 (ICAM-1) levels. In some embodiments, the high dose or an individual dose is an amount which increases the subject's HDL and/or ApoA-I levels by at least 25%, at least 30%, or at least 35% 2 to 4 hours after administration.

In some embodiments, the high dose is an amount effective to reduce serum levels of one or more inflammatory markers, for example, one or more of IL-6, C-reactive protein, D-dimer, ferritin, IL-8, GM-CSF, and MCP1 TNF-α. In some embodiments, the serum levels of the one or more inflammatory markers are reduced from an elevated range to a normal range, and/or reduced by at least 20%, at least 40%, or at least 60%.

The dose of a lipid binding protein-based complex (e.g., CER-001) administered to a subject (e.g., an individual dose which when aggregated with one or more other individual doses forms a high dose) can in some embodiments range from 4 to 40 mg/kg (e.g., 10 to 40 mg/kg) on a protein weight basis (e.g., 5, 10, 15, 20, 25, 30, 35, or 40 mg/kg or any range bounded by any two of the foregoing values, e.g., 10 to 20 mg/kg, 15 to 25 mg/kg, 20 to 40 mg/kg, 25 to 35 mg/kg, or 30 to 40 mg/kg). As used herein, the expression “protein weight basis” means that a dose of a lipid binding protein-based complex (e.g., CER-001) to be administered to a subject is calculated based upon the amount of ApoA-I in the lipid binding protein-based complex (e.g., CER-001) to be administered and the weight of the subject. For example, a subject who weighs 70 kg and is to receive a 20 mg/kg dose of CER-001 would receive an amount of CER-001 that provides 1400 mg of ApoA-I (70 kg×20 mg/kg).

In yet other aspects, a lipid binding protein-based complex (e.g., CER-001) can be administered on a unit dosage basis. The unit dosage used in the methods of the disclosure can in some embodiments vary from 300 mg to 4000 mg (e.g., 600 mg to 4000 mg) per administration (on a protein weight basis).

In particular embodiments, the dosage of a lipid binding protein-based complex (e.g., CER-001) is 600 mg to 3000 mg, 800 mg to 3000 mg, 1000 mg to 2400 mg, or 1000 mg to 2000 mg per administration (on a protein weight basis).

In some aspects, a high dose of a lipid binding protein-based complex (e.g., CER-001), e.g., the aggregate of multiple individual doses, is 600 mg to 40 g (on a protein weight basis). In particular embodiments, a high dose is 3 g to 35 g or 5 g to 30 g (on a protein weight basis).

A lipid binding protein-based complex (e.g., CER-001) is preferably administered as an IV infusion. For example, a stock solution of CER-001 can be diluted in normal saline such as physiological saline (0.9% NaCl) to a total volume between 125 and 250 ml. In some embodiments, subjects weighing less than 80 kg will have a total volume of 125 ml whereas subjects weighing at least 80 kg will have a total volume of 250 ml. In some embodiments, doses of CER-001 are administered in a total volume of 250 ml. A lipid binding protein-based complex (e.g., CER-001) may be administered over a period ranging from one-hour to 24-hours. Depending on the needs of the subject, administration can be by slow infusion with a duration of more than one hour (e.g., up to 2 hours or up to 24 hours), by rapid infusion of one hour or less, or by a single bolus injection. In an embodiment, a lipid binding protein-based complex (e.g., CER-001) is administered over a one-hour period, e.g., using an infusion pump at a fixed rate of 125 ml/hr or 250 ml/hr. In an embodiment, a dose of a lipid binding protein-based complex (e.g., CER-001) is administered as an infusion over a 24-hour period.

### 6.3.1. Induction Regimen

In one embodiment, induction regimens suitable for use in the methods of the disclosure entail administering multiple doses of a lipid binding protein-based complex (e.g., CER-001) over multiple consecutive days, e.g., three consecutive days.

In some embodiments, induction regimens suitable for use in the methods of the disclosure entail twice daily administration of a lipid binding protein-based complex (e.g., CER-001) such as twice daily administration on multiple consecutive days. Twice daily administration can comprise, for example, two doses approximately 12 hours apart or a morning dose and an evening dose (which may be more or less than 12 hours apart).

In an embodiment, the induction regimen comprises two doses of a lipid binding protein-based complex (e.g., CER-001) per day for 3 consecutive days.

A therapeutic dose of a lipid binding protein-based complex (e.g., CER-001) administered by infusion in the induction regimen can range from 4 to 40 mg/kg (e.g., 4 to 30 mg/kg) on a protein weight basis (e.g., 4, 5, 6, 7, 8, 9, 10, 12 15, 20, 25, 30 or 40 mg/kg, or any range bounded by any two of the foregoing values, e.g., 5 to 15 mg/kg, 10 to 20 mg/kg, or 15 to 25 mg/kg). In some embodiments, the dose of a lipid binding protein-based complex (e.g., CER-001) used in the induction regimen is 5 mg/kg. In some embodiments, the dose of a lipid binding protein-based complex (e.g., CER-001) used in the induction regimen is 10 mg/kg. In some embodiments, the dose of a lipid binding protein-based complex (e.g., CER-001) used in the induction regimen is 15 mg/kg. In some embodiments, the dose of a lipid binding protein-based complex (e.g., CER-001) used in the induction regimen is 20 mg/kg. In some embodiments, the induction regimen comprises six doses of a lipid binding protein-based complex (e.g., CER-001) administered over three days at a dose of 5 mg/kg, 10 mg/kg, 15 mg/kg or 20 mg/kg.

In yet other aspects, a lipid binding protein-based complex (e.g., CER-001) can be administered on a unit dosage basis. The unit dosage used in the induction phase can vary from 300 mg to 4000 mg (e.g., 300 mg to 3000 mg) (on a protein weight basis) per administration by infusion.

In particular embodiments, the dosage of a lipid binding protein-based complex (e.g., CER-001) used during the induction phase is 300 mg to 1500 mg, 400 mg to 1500 mg, 500 mg to 1200 mg, or 500 mg to 1000 mg (on a protein weight basis) per administration by infusion.

### 6.3.2. Consolidation Regimen

Consolidation regimens suitable for use in the methods of the disclosure entail administering one dose or multiple doses of a lipid binding protein-based complex (e.g., CER-001) following an induction regimen.

In one embodiment, the consolidation regimen comprises administering two doses of a lipid binding protein-based complex (e.g., CER-001). For example, the two doses can be administered approximately 12 hours apart, or administered as a morning dose and an evening dose (which may be more or less than 12 hours apart).

The dose(s) of a lipid binding protein-based complex (e.g., CER-001) in a consolidation regimen can in some embodiments be administered on day 6 of a dosing regimen that begins with an induction regimen on day 1. The dose(s) of a lipid binding protein-based complex (e.g., CER-001) in a consolidation regimen can in some embodiments be administered on day 4 of a dosing regimen that begins with an induction regimen on day 1. The dose(s) of a lipid binding protein-based complex (e.g., CER-001) in a consolidation regimen can in some embodiments be administered on day 5 of a dosing regimen that begins with an induction regimen on day 1. The dose(s) of a lipid binding protein-based complex (e.g., CER-001) in a consolidation regimen can in some embodiments be administered on day 7 of a dosing regimen that begins with an induction regimen on day 1.

A therapeutic dose of a lipid binding protein-based complex (e.g., CER-001) administered by infusion in the consolidation regimen can range from 4 mg/kg to 40 mg/kg (e.g., 4 to 30 mg/kg) on a protein weight basis (e.g., 4, 5, 6, 7, 8, 9, 10, 12, 15, 20, 25, 30, or 40 mg/kg, or any range bounded by any two of the foregoing values, e.g., 5 to 15 mg/kg, 10 to 20 mg/kg, or 15 to 25 mg/kg). In some embodiments, the dose of a lipid binding protein-based complex (e.g., CER-001) used in the consolidation regimen is 5 mg/kg. In some embodiments, the dose of a lipid binding protein-based complex (e.g., CER-001) used in the consolidation regimen is 10 mg/kg. In some embodiments, the dose of a lipid binding protein-based complex (e.g., CER-001) in the consolidation regimen is 15 mg/kg. In some embodiments, the dose of a lipid binding protein-based complex (e.g., CER-001) used in the consolidation regimen is 20 mg/kg. In some embodiments, the consolidation regimen comprises two doses of a lipid binding protein-based complex (e.g., CER-001) administered on one day at a dose of 5 mg/kg, 10 mg/kg, 15 mg/kg or 20 mg/kg.

In yet other aspects, a lipid binding protein-based complex (e.g., CER-001) can be administered on a unit dosage basis. The unit dosage used in the consolidation phase can vary from 300 mg to 4000 mg (e.g., 300 mg to 3000 mg) (on a protein weight basis) per administration by infusion.

In particular embodiments, the dosage of a lipid binding protein-based complex (e.g., CER-001) used during the consolidation phase is 300 mg to 1500 mg, 400 mg to 1500 mg, 500 mg to 1200 mg, or 500 mg to 1000 mg (on a protein weight basis) per administration by infusion.

The lipid binding protein-based complex (e.g., CER-001) can be administered during the consolidation phase in the same manner as described in Section 6.3, e.g., as an IV infusion over a one-hour period.

### 6.4. Combination Therapies

A lipid binding protein-based complex (e.g., CER-001) can be administered to a subject as described herein as a monotherapy or a part of a combination therapy regimen. For example, a combination therapy may comprise a lipid binding protein-based complex (e.g., CER-001) in combination with a standard of care treatment for sepsis and/or AKI. See, e.g., Rhodes et al., 2017, Intensive Care Med 43:304-377; Dugar et al., 2020, Cleveland Clinic Journal of Medicine 87(1):53-64.

In some embodiments, the subject is treated with a lipid binding protein-based complex (e.g., CER-001) in combination with fluid replacement therapy. In some embodiments, the subject is treated with a lipid binding protein-based complex (e.g., CER-001) in combination with an antimicrobial. In some embodiments, the subject is treated with a lipid binding protein-based complex (e.g., CER-001) in combination with an antibiotic (e.g., ceftriaxone, meropenem, ceftazidime, cefotaxime, cefepime, piperacillin and tazobactam, ampicillin and sulbactam, imipenem and cilastatin, levofloxacin, clindamycin, or an oxazolidinone class antibiotic such as linezolid or tedizolid). In some embodiments, the subject is treated with a lipid binding protein-based complex (e.g., CER-001) in combination with an antiviral. In some embodiments, the subject is treated with a lipid binding protein-based complex (e.g., CER-001) in combination with a medication that raises blood pressure (e.g., norepinephrine or epinephrine). In some embodiments, the subject is treated with a lipid binding protein-based complex (e.g., CER-001) in combination with an immunosuppressant such as tacrolimus or everolimus.

A combination therapy regimen can in some embodiments comprise one or more anti-IL-6 agents and/or one or more other agents for treating CRS such as corticosteroids (e.g., methylprednisolone and/or dexamethasone). Exemplary anti-IL6 agents include tocilizumab, siltuximab, olokizumab, elsilimomab, BMS-945429, sirukumab, levilimab, and CPSI-2364. In some embodiments, a lipid binding protein-based complex (e.g., CER-001) is administered in combination with tocilizumab. Subjects who have or have had a COVID-19 infection can be treated with a lipid binding protein-based complex (e.g., CER-001) in combination with one or more additional therapies such as antibodies from recovered COVID-19 patients, antibodies against the spike protein of COVID-19 (e.g., casirimivab, imdevimab), one or more antiviral agents (e.g., lopinavir, remdesivir, danoprevir, galidesivir, darunavir, ritonavir), chloroquine, hydroxychloroquine, azithromycin, an interferon (e.g., an interferon alpha or an interferon beta, each of which can be pegylated), or a combination thereof.

In certain embodiments, an antihistamine (e.g., diphenhydramine, cetirizine, fexofenadine, or loratadine) can be administered before administration of a lipid binding protein-based complex (e.g., CER-001). The antihistamine can reduce the likelihood of allergic reactions.

### 7. EXAMPLES

### 7.1. Example 1: CER-001 Therapy for COVID-19

The SARS-CoV-2 virus can promote life-threatening hyperinflammatory states in at-risk patients. Remodeling of the lipid profile, including a dramatic decrease in the serum levels of apolipoprotein-A-1 (ApoA-1), is the hallmark of critical COVID-19. ApoA-I can reduce lung inflammation, modulates innate and adaptative immunity and prevents endothelial dysfunction and blood coagulation. This Example describes a compassionate-access trial in four subjects with COVID-19 cytokine storm that progressed despite standard-of-care therapy. To raise ApoA-I to normal levels, subjects received 2-4 infusions of CER-001 (10 mg/kg each). Injections were well tolerated with no serious adverse events. Three patients had rapid improvement and were discharged from the hospital 3-4 days after CER-001 infusions. In the fourth, who received CER-001 while on mechanical ventilation, a transient improvement was followed by bacterial pneumonia-related worsening. This study provides early safety and proof-of-concept data for treatment of patients with virus-induced cytokine storm using lipid binding protein-based complexes such as CER-001.

### 7.1.1. Materials and Methods

### 7.1.1.1. Case Histories

Subject 1 was a 52-year-old male with a history of IgA vasculitis, diabetes mellitus and ischemic heart disease, and who had received a kidney transplant in 2018. He had been given 3 doses of mRNA COVID-19 vaccine but developed only weak anti-SARS-CoV-2 immunity (anti-spike antibodies 15.5 BAU/mL). He developed symptoms of COVID-19 (fever, diarrhea, and dyspnea) and was admitted to the transplantation ward eight days later. Oxygen saturation was 92%, and in room air and oxygen supplementation was started (1 L/min). A chest CT scan showed bilateral interstitial lung disease compatible with COVID-19 (parenchyma extension 25%), and nasopharyngeal PCR identified a SARS-CoV-2 (variant-of-concern (VOC)) Delta. Blood tests showed a hyperinflammatory state (ferritin 5,037 μg/L, C-reactive protein 34 mg/L), liver test abnormalities (AST and ALT 2.5- and 3.5 times the upper limit normal (ULN) values, respectively) and thrombocytopenia. Tacrolimus was administered, mycophenolate mofetil was withdrawn, and dexamethasone was introduced (6 mg/day) with antibiotics. On day 2, blood tests showed pancytopenia, and progression of the hyperinflammatory state (ferritin 6,870 μg/L, C-reactive protein 55 mg/L). The subject received one infusion of the monoclonal anti-IL-6R antibody tocilizumab (8 mg/kg i.v.) and one infusion of neutralizing monoclonal anti-SARS-Cov2 antibodies (casirimivab/imdevimab). On day 4, the subject's ferritin increased to 19,219 μg/L, the subject's AST and ALT increased to 17 and 14 times the ULN values, respectively, and the subject's arterial lactates were at 2.7 mmol/L. Bone marrow aspirate showed features of hemophagocytosis. Blood PCR of SARS-CoV-2 was weakly positive. Worsening hypoxia required increased oxygenation (4 L/min; PaO2 62 mmHg) and a CT scan showed progressive lung lesions typical of COVID-19 (50% of the parenchyma). Despite increasing dexamethasone to 10 mg/day, serum triglycerides and ferritin increased to 3.2 mmol/L and 27,394 μg/L, respectively, on day 6.

Subject 2 was a 38-year-old female with history of systemic lupus erythematosus and being overweight, and who had received a kidney transplant in 2011. She had been given three doses of mRNA COVID-19 vaccines but developed no anti-SARS-CoV-2 immunity. She developed symptoms of COVID-19 (cough, chills, diarrhea and fever) and was admitted to the transplantation ward ten days later. Nasopharyngeal PCR identified SARS-CoV2 VOC Omicron. Upon admission, SaO2 was 94% while receiving 9 L/min of oxygen with a facial mask. A chest CT scan showed typical lesions of COVID-19 (extension 50%). Blood tests showed hepatitis with cytolysis and cholestasis (7 to 10 times the ULN values, respectively), acute kidney injury (KDIGO stage 1) and hyperinflammation (ferritin 2,000 μg/L, C-reactive protein 107 mg/L). High-flow oxygen supplementation, awake prone position, dexamethasone (10 mg/day), tocilizumab (8 mg/kg once) and antibiotics were started. Everolimus was withdrawn and tacrolimus was administered. On day 4, despite full-code therapy, high-flow oxygen supplementation was still required and the subject's hyperinflammatory state worsened (ferritin 2,800 μg/L).

Subject 3 was a 47-year-old female with history of diabetes mellitus, adrenal Cushing's syndrome, hypertension, and end-stage kidney disease requiring chronic kidney replacement therapy since 2020. She did not receive anti-SARS-CoV-2 vaccination and had no anti-SARS-CoV-2 immunity at the admission to the hospital. She developed symptoms of COVID-19 (cough, dyspnea, abdominal pain, and fever) and was admitted to the hospital four days later. Nasopharyngeal PCR identified SARS-CoV2 VOC Omicron. Chest CT scan showed mild to moderate lung lesions typical of COVID-19 (10-25%). She did not require oxygen supplementation. Blood tests showed hyperinflammatory syndrome (ferritin 4,350 μg/L and C-reactive protein 55 mg/L) with moderate increase in AST and ALT (2- and 1.5 times the ULN values, respectively), and mild thrombocytopenia and anemia. Dexamethasone (6 mg/day) was introduced. On day 3, hyperferritinemia (4,142 μg/L) and liver test abnormalities persisted, and she developed encephalopathy leading to admission to the intensive care unit.

Subject 4 was a 59-year-old male with a history of hepatitis B, liver transplantation in 2006, HHV8-negative Kaposi's sarcoma (complete remission), and end-stage kidney disease requiring chronic kidney replacement therapy since 2020. He had received 3 doses of mRNA COVID-19 vaccines but developed no anti-SARS-CoV-2 antibodies. Owing to familial exposure to SARS-CoV2, nasopharyngeal PCR was performed, identifying the VOC Omicron. He developed symptoms of COVID-19 (asthenia) but initially had no respiratory symptoms. Chest CT scan showed mild to moderate lung lesions typical of COVID-19 (10-25%). Two days later, dyspnea, cough, and fever developed. Upon admission, PaO2 was 54 mmHg in room air, respiratory rate was 30 cycles/min, and body temperature was 38.5° C. Blood tests showed hyperferritinemia (1,223 μg/L) and leukopenia (1,080 cells/mm³). A CT scan showed progression of lung lesions (25-50%). Oxygen supplementation, dexamethasone (10 mg/day), tocilizumab (8 mg/kg, once), antibiotics, and fresh frozen plasma from convalescent subjects were administered. Mycophenolate mofetil was withdrawn, and tacrolimus was administered. On day 5, acute respiratory failure developed requiring orotracheal intubation and mechanical ventilation with neuromuscular blocking. Blood tests showed a hyperinflammatory state (ferritin 4,535 μg/L) with increased AST and ALT (3 times the ULN values). At that time, the bronchoalveolar fluid culture was negative, suggestive of critical COVID-19 only. PaO2 to FiO2 ratio was 150 to 180. Antibiotics were administered.

### 7.1.1.2. Dosing

CER-001 was administered intravenously to Subject 1 over a period of 0.5 to 1 hour at a dose of 10 mg/kg at hours 0 and 12. CER-001 was administered intravenously to Subjects 2-4 over a period of 0.5 to 1 hour at a dose of 10 mg/kg at hours 0, 12, 24, and 48. Each dose of CER-001 was preceded by anti-histaminic prophylaxis with hydroxyzine (50 mg 172 i.v.). All subjects were also administered dexamethasone.

### 7.1.2. Results and Discussion

### 7.1.2.1. General Safety

Subjects 1, 2 and 3 did not develop any serious adverse events. Subject 4 developed two episodes of ventilation-associated pneumonia (VAP; Klebsiella Pneumoniae and Aspergillus Fumigatus plus Mucormycosis) and one bacteremia (Staphylococcus Haemolyticus).

### 7.1.2.2. Bioefficacy: Lipid Profiles

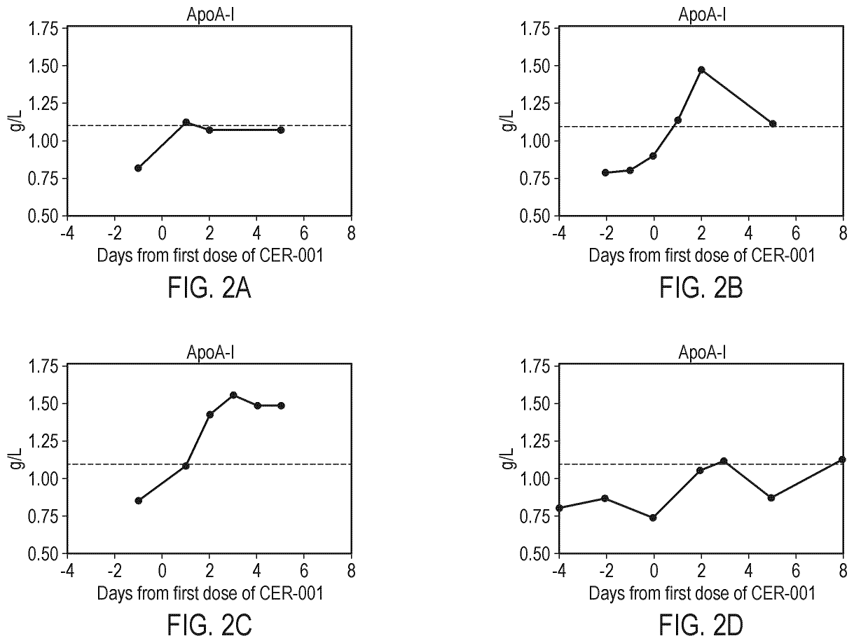

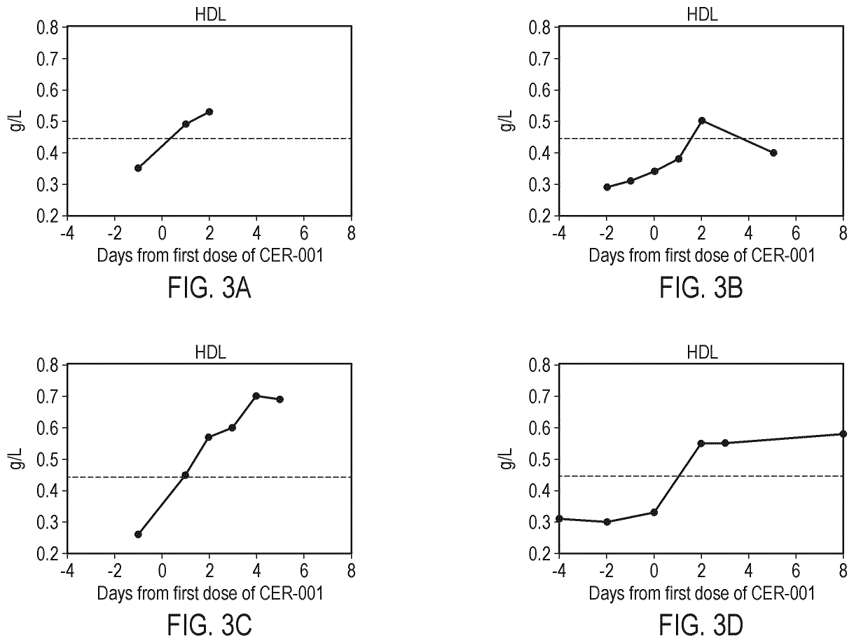

All four subjects had very low serum levels of ApoA-I (range 0.74 to 0.79 mg/L, normal value >1.1 g/L) (FIG. 2A-FIG. 2D) and HDL (range 0.26 to 0.35, normal values >0.45 g/L) (FIG. 3A-FIG. 3D), and high serum levels of triglycerides (range 2.16 to 3.4 g/L, normal value<1.5 g/L) when CER-001 was first administered. Following treatment with CER-001, ApoA-I and HDL levels normalized in all subjects at day 2 but remained in the lower range of the normal values in the most inflammatory subjects. In Subject 4, who developed ventilator associated pneumonia three days after the start of CER-001, ApoA-I subsequently decreased below the normal values.

### 7.1.2.3. Inflammation Kinetics

At baseline, IL-1p was normal in all individuals, IL-6 was increased in the three subjects who previously received tocilizumab and was normal in the fourth subject (3.3 to 1,295 μg/mL) and TNF-α was moderately increased (9.7 to 42.1 μg/mL). IL-8 was the only inflammatory cytokine universally increased (>10 μg/mL; 14.8 to 64.5 μg/mL). Following CER-001 administration, IL-8 normalized in Subjects 1, 2 and 3. In subject 4, IL-8 decreased immediately after the injections and re-increased at the time of a ventilator-associated pneumonia. Serum levels of ferritin decreased from 6,616±8,696 to 1,712±815 μg/L six days after the start of CER-001. The administration of anti-IL6R antibodies before CER-001 in 3 out of 4 subjects precluded the analysis of C-reactive protein. Body temperature remained below 37.5° in all subjects.

### 7.1.2.4. Clinical Outcomes

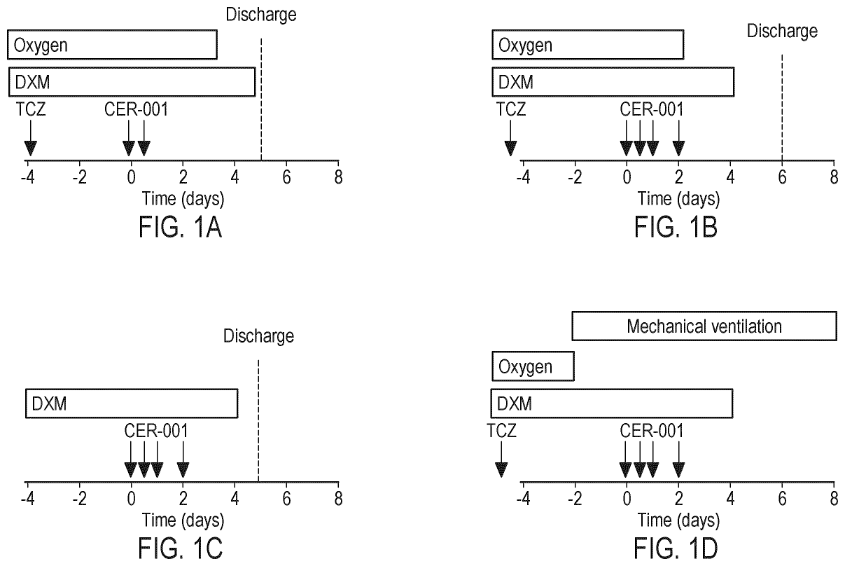

CER-001 administration was followed by rapid improvement of the clinical condition of Subjects 1, 2 and 3 allowing them to be discharged from the hospital 3 to 4 days after the CER-001 infusions (FIG. 1A-FIG. 1D). In Subjects 1 and 2, oxygen supplementation was withdrawn 2 and 3 days after administration. In subject 3, confusion resolved within 2 days. In these 3 subjects, inflammatory parameters, liver tests and blood cell counts improved until discharge. Subject 4 had been receiving mechanical ventilation for 3 days when CER-001 was introduced. After a first phase of improvement (neuromuscular blockers withdrawal and sedation lightening) for three days, he secondary developed several ventilator-associated pneumonia and ultimately died one month later.

### 7.1.2.5. Discussion

In addition to a very good acute tolerance of CER-001, a rapid improvement in respiratory status, a decrease in inflammatory parameters, and the normalization of blood cell counts paralleling the normalization of ApoA-I levels after CER-001 was observed in three out of the four subjects. Whereas they had been developing critical COVID-19-related cytokine storm, they were able to be discharged home, without oxygen support, as soon as 3 to 4 days after CER-001 infusions. Following administration of CER-001, a rapid decrease in IL-8 in the three subjects with a favorable outcome, paralleling the clinical and biological improvement, was observed. In Subject 4, after a first phase of clinical improvement accompanied by ApoA-I normalization and IL-8 decrease, ventilation-associated pneumonia and clinical deterioration were accompanied by C-reactive protein and IL-8 increase, as well as ApoA-I decrease. In this study, subjects received four infusions of CER-001 (10 mg/kg), but the use of higher dose for the first injections (e.g., 15 mg/kg) may help to reach the optimal concentrations of ApoA-I and non-oxidized HDL more rapidly to achieve maximal therapeutic effects.

### 8. SPECIFIC EMBODIMENTS

Various aspects of the present disclosure are described in the embodiments set forth in the following numbered paragraphs.

1. A method of treating a subject with an acute condition, comprising administering to the subject in need thereof a high dose of a lipid binding protein-based complex, optionally wherein the acute condition comprises acute inflammation.

2. The method of embodiment 1, wherein the high dose is administered over a period of one day to approximately two weeks, optionally wherein the high dose is administered over a period of one day, two days, three days, four days, five days, six days, seven days, eight days, nine days, 10 days, eleven days, 12 days, 13 days, 14 days or 15 days.

3. The method of embodiment 1 or embodiment 2, wherein the high dose is the aggregate of two to ten individual doses, optionally wherein the high dose is an aggregate of three, four, five, six, seven, eight, nine or 10 individual doses.

4. The method of embodiment 3, wherein a plurality of individual doses are administered daily or twice daily.

5. The method of embodiment 3 or embodiment 4, wherein a plurality of individual doses are administered two to three days apart.

6. The method of embodiment 3, wherein a plurality of individual doses are administered no more than one day apart.

7. The method of embodiment 6, which comprises administering two or more individual doses approximately 12 hours apart.

8. The method of embodiment 7, which comprises administering two individual doses approximately 12 hours apart.

9. The method of embodiment 7, which comprises administering three individual doses approximately 12 hours apart.

10. The method of embodiment 8 or embodiment 9, which further comprises administering an individual dose approximately one day later.

11. The method of embodiment 3, which comprises administering three individual doses approximately 12 hours apart and a fourth individual dose approximately one day later.

12. The method of embodiment 1, wherein the high dose is administered as a single individual dose.

13. The method of embodiment 1, wherein the high dose is the aggregate of two individual doses administered in one day.

14. The method of embodiment 13, wherein the two individual doses are administered approximately 12 hours apart.

15. The method of any one of embodiments 3 to 14, wherein each individual dose is effective to increase the subject's HDL levels.

16. The method of embodiment 15, wherein the high dose is effective to increase the subject's serum HDL levels to normal (e.g., >0.45 g/L).

17. The method of embodiment 15, wherein each individual dose is effective to increase the subject's HDL levels by at least 25%, at least 30% or at least 35% 2-4 hours after administration.

18. The method of embodiment 17, wherein each individual dose is effective to increase the subject's HDL levels by at least 25%, at least 30% or at least 35% 2 hours after administration.

19. The method of embodiment 17, wherein each individual dose is effective to increase the subject's HDL levels by at least 25%, at least 30% or at least 35% 3 hours after administration.

20. The method of embodiment 17, wherein each individual dose is effective to increase the subject's HDL levels by at least 25%, at least 30% or at least 35% 4 hours after administration.

21. The method of any one of embodiments 3 to 20, wherein each individual dose is effective to increase the subject's ApoA-1 levels.

22. The method of embodiment 21, wherein the high dose is effective increase the subject's serum ApoA-1 levels to normal (e.g., >1.1 g/L).

23. The method of embodiment 21, wherein each individual dose is effective to increase the subject's ApoA-1 levels by at least 25%, at least 30% or at least 35% 2-4 hours after administration.

24. The method of embodiment 22, wherein each individual dose is effective to increase the subject's ApoA-1 levels by at least 25%, at least 30% or at least 35% 2 hours after administration.

25. The method of embodiment 22, wherein each individual dose is effective to increase the subject's ApoA-1 levels by at least 25%, at least 30% or at least 35% 3 hours after administration.

26. The method of embodiment 22, wherein each individual dose is effective to increase the subject's ApoA-1 levels by at least 25%, at least 30% or at least 35% 4 hours after administration.

27. The method of any one of embodiments 1 to 26, wherein the high dose is effective to improve the subject's vascular endothelial function, optionally wherein vascular endothelial function is measured by circulating VCAM-1 and/or ICAM-1.

28. The method of any one of embodiments 1 to 27, wherein the high dose is effective to reduce serum levels of one or more inflammatory markers in the subject.

29. The method of embodiment 28, wherein the high dose is effective to reduce serum levels of interleukin-6 (“IL-6”).

30. The method of embodiment 28 or embodiment 29, wherein the high dose is effective to reduce serum levels of C-reactive protein.

31. The method of any one of embodiments 28 to 30, wherein the high dose is effective to reduce serum levels of D-dimer.

32. The method of any one of embodiments 28 to 31, wherein the high dose is effective to reduce serum levels of ferritin.

33. The method of any one of embodiments 28 to 32, wherein the high dose is effective to reduce serum levels of interleukin 8 (IL-8).

34. The method of any one of embodiments 28 to 32, wherein the high dose is effective to normalize serum levels of interleukin 8 (IL-8).

35. The method of any one of embodiments 28 to 34, wherein the high dose is effective to reduce serum levels of granulocyte-macrophage colony stimulating factor (GM-CSF).

36. The method of any one of embodiments 28 to 35, wherein the high dose is effective to reduce serum levels of monocyte chemoattractant protein (MCP) 1.

37. The method of any one of embodiments 28 to 36, wherein the high dose is effective to reduce serum levels of tumor necrosis factor α (TNF-α).

38. The method of any one of embodiments 28 to 37, wherein the high dose is effective to reduce serum levels of the one or more inflammatory markers from an elevated range to a normal range.

39. The method of any one of embodiments 28 to 38, wherein the high dose is effective to reduce serum levels of the one or more inflammatory markers by at least 20%, by at least 40% or by at least 60%.

40. The method of any one of embodiments 1 to 39, wherein the subject has a viral infection.

41. The method of embodiment 40, wherein the viral infection is a coronavirus infection.

42. The method of embodiment 41, wherein the coronavirus infection is COVID-19.

43. The method of any one of embodiments 1 to 42, wherein the subject has CRS or is at risk of CRS.

44. The method of embodiment 43, wherein the subject has CRS.

45. The method of embodiment 44, wherein the subject has CRS secondary to an infection.

46. The method of embodiment 45, wherein the infection is a viral infection.

47. The method of embodiment 46, wherein the viral infection is a coronavirus infection.

48. The method of embodiment 47, wherein the coronavirus is COVID-19.

49. The method of embodiment 46, wherein the viral infection is influenza infection.

50. The method of embodiment 44, wherein the subject has CRS caused by immunotherapy.

51. The method of embodiment 50, wherein the immunotherapy comprises antibody therapy.

52. The method of embodiment 50, wherein the immunotherapy comprises chimeric antigen receptor (CAR) T cell therapy.

53. The method of any one of embodiments 50 to 52, wherein the lipid binding protein-based complex is administered before the immunotherapy begins.

54. The method of any one of embodiments 50 to 53, wherein the lipid binding protein-based complex is administered concurrently with the immunotherapy.

55. The method of any one of embodiments 50 to 54, wherein the lipid binding protein-based complex is administered after the immunotherapy ends.

56. The method of embodiment 43, wherein the subject is at risk of CRS.

57. The method of embodiment 56, wherein the subject is at risk of CRS due to an infection.

58. The method of embodiment 57, wherein the infection is a viral infection.

59. The method of embodiment 58, wherein the viral infection is a coronavirus infection.

60. The method of embodiment 59, wherein the coronavirus is COVID-19.

61. The method of embodiment 58, wherein the viral infection is influenza infection.

62. The method of embodiment 56, wherein the subject is at risk of CRS due to immunotherapy.

63. The method of embodiment 62, wherein the immunotherapy comprises antibody therapy.

64. The method of embodiment 62, wherein the immunotherapy comprises chimeric antigen receptor (CAR) T cell therapy.

65. The method of any one of embodiments 62 to 64, wherein the lipid binding protein-based complex is administered before the immunotherapy begins.

66. The method of any one of embodiments 62 to 65, wherein the lipid binding protein-based complex is administered concurrently with the immunotherapy.

67. The method of any one of embodiments 62 to 66, wherein the lipid binding protein-based complex is administered after the immunotherapy ends.

68. The method of any one of embodiments 1 to 42, wherein the subject has or is at risk of developing sepsis.

69. The method of embodiment 68, wherein the sepsis is associated with a gram-negative bacterial infection.

70. The method of embodiment 68, wherein the sepsis is associated with a gram-positive bacterial infection.

71. The method of any one of embodiments 68 to 70, wherein the subject has an intra-abdominal cavity infection.

72. The method of any one of embodiments 68 to 70, wherein the subject has urosepsis.

73. The method of any one of embodiments 68 to 72, wherein the high dose is effective to reduce the severity of the sepsis.

74. The method of any one of embodiments 1 to 73, wherein the high dose is effective to reduce the likelihood that the subject will develop acute kidney injury (AKI).

75. The method of any one of embodiments 1 to 74, wherein the high dose is effective to delay the onset of AKI.

76. The method of any one of embodiments 1 to 74, wherein the high dose is effective to prevent AKI.

77. The method of any one of embodiments 1 to 73, wherein the subject has or is at risk of developing acute kidney injury (AKI).

78. The method of embodiment 77, wherein the AKI is sepsis-related AKI.

79. The method of embodiment 77, wherein the AKI is ischemia/reperfusion AKI.

80. The method of embodiment 77, wherein the AKI is cardiac surgery-associated AKI.

81. The method of embodiment 77, wherein the AKI is hepatorenal syndrome (HRS) AKI.

82. The method of embodiment 81, wherein the HRS is type 1 HRS.

83. The method of embodiment 81, wherein the HRS is type 2 HRS.

84. The method of any one of embodiments 77 to 83, wherein the subject has AKI.

85. The method of embodiment 84, wherein the AKI is secondary to a viral infection, optionally wherein the viral infection is COVID-19.

86. The method of embodiment 84 or embodiment 85, wherein the high dose is effective to reduce the severity of the AKI.

87. The method of any one of embodiments 77 to 81, wherein the subject is at risk for AKI.

88. The method of embodiment 87, wherein the subject has sepsis.

89. The method of embodiment 88, wherein the sepsis is associated with a gram-negative bacterial infection.

90. The method of embodiment 88, wherein the sepsis is associated with a gram-positive bacterial infection.

91. The method of any one of embodiments 88 to 90, wherein the subject has an intra-abdominal cavity infection.

92. The method of any one of embodiments 88 to 90, wherein the subject has urosepsis.

93. The method of embodiment 87, wherein the subject has a viral infection, optionally wherein the viral infection is COVID-19.

94. The method of embodiment 87, wherein the subject has undergone cardiac surgery.

95. The method of embodiment 87, wherein the subject has acute liver disease.

96. The method of embodiment 87, wherein the subject has chronic liver disease.

97. The method of any one of embodiments 87 to 96, wherein the high dose is effective to reduce the likelihood that the subject will develop AKI.

98. The method of any one of embodiments 87 to 97, wherein the high dose is effective to delay the onset of AKI.

99. The method of any one of embodiments 87 to 97, wherein the high dose is effective to prevent AKI.

100. The method of any one of embodiments 87 to 98, wherein if the subject develops AKI, the high dose is effective to reduce the severity of the AKI.

101. The method of any one of embodiments 68 to 100, wherein the subject has a total SOFA score of 2 to 24 prior to administration of the lipid binding protein-based complex.

102. The method of embodiment 101, wherein the subject has a total SOFA score of 2 to 5 prior to administration of the lipid binding protein-based complex.

103. The method of embodiment 101, wherein the subject has a total SOFA score of 2 to 10 prior to administration of the lipid binding protein-based complex.

104. The method of embodiment 101, wherein the subject has a total SOFA score of 5 to 15 prior to administration of the lipid binding protein-based complex.

105. The method of embodiment 101, wherein the subject has a total SOFA score of 8 to 13 prior to administration of the lipid binding protein-based complex

106. The method of embodiment 101, wherein the subject has a total SOFA score of 10 to 15 prior to administration of the lipid binding protein-based complex.

107. The method of embodiment 101, wherein the subject has a total SOFA score of 10 to 20 prior to administration of the lipid binding protein-based complex.

108. The method of embodiment 101, wherein the subject has a total SOFA score of 15 to 24 prior to administration of the lipid binding protein-based complex.

109. The method of any one of embodiments 1 to 108, wherein the subject has an endotoxin activity level of >0.6 prior to administration of the lipid binding protein-based complex.

110. The method of any one of embodiments 1 to 109, wherein the high dose is effective to reduce the subject's endotoxin activity level.

111. The method of any one of embodiments 1 to 110, wherein the lipid binding protein-based complex is a reconstituted HDL or HDL mimetic.

112. The method of any one of embodiments 1 to 110, wherein the lipid binding protein-based complex is an Apomer or a Cargomer.

113. The method of any one of embodiments 1 to 112, wherein the lipid binding protein-based complex comprises a sphingomyelin.

114. The method of any one of embodiments 1 to 113, wherein the lipid binding protein-based complex comprises a negatively charged lipid.

115. The method of embodiment 114, wherein the negatively charged lipid is 1,2-dipalmitoyl-sn-glycero-3-[phospho-rac-(1-glycerol) (DPPG) or a salt thereof.

116. The method of embodiment 111, wherein the lipid binding protein-based complex is CER-001, CSL-111, CSL-112, CER-522 or ETC-216.

117. The method of embodiment 116, wherein the lipid binding protein-based complex is CER-001.

118. The method of any one of embodiments 1 to 117, wherein the lipid binding protein-based complex is administered systemically, optionally by infusion.

119. The method of any one of embodiments 1 to 118, wherein the lipid binding protein-based complex is administered until serum levels of one or more inflammatory markers are reduced.

120. The method of embodiment 119, wherein the lipid binding protein-based complex is administered until serum levels of one or more inflammatory markers are reduced to a normal range(s).

121. The method of embodiment 119, wherein the lipid binding protein-based complex is administered until serum levels of one or more inflammatory markers are reduced below a baseline level(s) for the one or more inflammatory markers measured prior to lipid binding protein-based complex administration.

122. The method of any one of embodiments 1 to 121, wherein each individual dose of the lipid binding protein-based complex administered is 4-40 mg/kg (on a protein weight basis).

123. The method of embodiment 122, wherein each individual dose of the lipid binding protein-based complex is 4-30 mg/kg (on a protein weight basis).

124. The method of embodiment 122, wherein each individual dose of the lipid binding protein-based complex is 15-25 mg/kg (on a protein weight basis).

125. The method of embodiment 122, wherein each individual dose of the lipid binding protein-based complex is 10-30 mg/kg (on a protein weight basis).

126. The method of embodiment 122, wherein each individual dose of the lipid binding protein-based complex is 10-20 mg/kg (on a protein weight basis).

127. The method of embodiment 122, wherein each individual dose of the lipid binding protein-based complex is 5 mg/kg (on a protein weight basis).

128. The method of embodiment 122, wherein each individual dose of the lipid binding protein-based complex is 10 mg/kg (on a protein weight basis).

129. The method of embodiment 122, wherein each individual dose of the lipid binding protein-based complex is 15 mg/kg (on a protein weight basis).

130. The method of embodiment 122, wherein each individual dose of the lipid binding protein-based complex is 20 mg/kg (on a protein weight basis).

131. The method of embodiment 122, wherein each individual dose of the lipid binding protein-based complex is 5 to 15 mg/kg (on a protein weight basis).

132. The method of embodiment 122, wherein each individual dose of the lipid binding protein-based complex is 10 to 20 mg/kg (on a protein weight basis).

133. The method of embodiment 122, wherein each individual dose of the lipid binding protein-based complex is 15 to 25 mg/kg (on a protein weight basis).

134. The method of any one of embodiments 1 to 133, wherein the high dose is administered according to an induction regimen, optionally followed by a consolidation regimen.

135. The method of embodiment 134, wherein the induction regimen comprises administering the lipid binding protein-based complex once daily or twice daily.

136. The method of embodiment 134 or embodiment 135, wherein the consolidation regimen comprises administering the lipid binding protein-based complex once daily or once every two days.

137. The method of any one of embodiments 1 to 136, wherein the subject is not treated with a maintenance regimen.

138. The method of any one of embodiments embodiment 134 to 137, wherein the consolidation regimen comprises administering one or more doses of the lipid binding protein-based complex to the subject one or more days after administration of the final dose of the induction regimen.

139. The method of embodiment 138, wherein the first dose of the lipid binding protein-based complex administered during the consolidation regimen is administered two or more days after administration of the final dose of the induction regimen.

140. The method of embodiment 138, wherein the first dose of the lipid binding protein-based complex administered during the consolidation regimen is administered three or more days after administration of the final dose of the induction regimen.

141. The method of embodiment 140, wherein the first dose of the lipid binding protein-based complex administered during the consolidation regimen is administered three days after administration of the final dose of the induction regimen.

142. The method of any one of embodiments 134 to 141, which comprises an induction regimen comprising twice daily administration of the lipid binding protein-based complex on days 1, 2, and 3 and a consolidation regimen comprising two doses of the lipid binding protein-based complex on day 6.

143. The method of any one of embodiments 134 to 142, wherein each individual dose of the lipid binding protein-based complex administered in the induction regimen is 4-40 mg/kg (on a protein weight basis).

144. The method of any one of embodiments 134 to 143, wherein each individual dose of the lipid binding protein-based complex administered in the induction regimen is 4-30 mg/kg (on a protein weight basis).

145. The method of any one of embodiments 134 to 143, wherein each individual dose of the lipid binding protein-based complex administered in the induction regimen is 15-25 mg/kg (on a protein weight basis).

146. The method of any one of embodiments 134 to 143, wherein each individual dose of the lipid binding protein-based complex administered in the induction regimen is 10-30 mg/kg (on a protein weight basis).

147. The method of any one of embodiments 134 to 143, wherein each individual dose of the lipid binding protein-based complex administered in the induction regimen is 10-20 mg/kg (on a protein weight basis).

148. The method of any one of embodiments 134 to 143, wherein each individual dose of the lipid binding protein-based complex administered in the induction regimen is 5 mg/kg (on a protein weight basis).

149. The method of any one of embodiments 134 to 143, wherein each individual dose of the lipid binding protein-based complex administered in the induction regimen is 10 mg/kg (on a protein weight basis).

150. The method of any one of embodiments 134 to 143, wherein each individual dose of the lipid binding protein-based complex administered in the induction regimen is 15 mg/kg (on a protein weight basis).

151. The method of any one of embodiments 134 to 143, wherein each individual dose of the lipid binding protein-based complex administered in the induction regimen is 20 mg/kg (on a protein weight basis).

152. The method of any one of embodiments 134 to 151, wherein the dose of the lipid binding protein-based complex administered in the consolidation regimen is 5 to 15 mg/kg (on a protein weight basis).

153. The method of any one of embodiments 134 to 151, wherein the dose of the lipid binding protein-based complex administered in the consolidation regimen is 10 to 20 mg/kg (on a protein weight basis).

154. The method of any one of embodiments 134 to 151, wherein the dose of the lipid binding protein-based complex administered in the consolidation regimen is 15 to 25 mg/kg (on a protein weight basis).

155. The method of any one of embodiments 134 to 151, wherein the dose of the lipid binding protein-based complex administered in the consolidation regimen is 5 mg/kg (on a protein weight basis).

156. The method of any one of embodiments 134 to 151, wherein the dose of the lipid binding protein-based complex administered in the consolidation regimen is 10 mg/kg (on a protein weight basis).

157. The method of any one of embodiments 134 to 151, wherein the dose of the lipid binding protein-based complex administered in the consolidation regimen is 15 mg/kg (on a protein weight basis).

158. The method of any one of embodiments 1 to 157, wherein each individual dose of the lipid binding protein-based complex administered is 300 mg to 4000 mg (on a protein weight basis).

159. The method of embodiment 158, wherein each individual dose of the lipid binding protein-based complex administered is 300 mg to 3000 mg (on a protein weight basis).

160. The method of embodiment 158, wherein each individual dose of the lipid binding protein-based complex administered is 300 mg to 1500 mg (on a protein weight basis).

161. The method of embodiment 158, wherein each individual dose of the lipid binding protein-based complex administered is 400 mg to 4000 mg (on a protein weight basis).

162. The method of embodiment 158, wherein each individual dose of the lipid binding protein-based complex administered is 400 mg to 1500 mg (on a protein weight basis).

163. The method of embodiment 158, wherein each individual dose of the lipid binding protein-based complex administered is 500 mg to 1200 mg (on a protein weight basis).

164. The method of embodiment 158, wherein each individual dose of the lipid binding protein-based complex administered is 500 mg to 1000 mg (on a protein weight basis).

165. The method of embodiment 158, wherein each individual dose of the lipid binding protein-based complex administered is 600 mg to 3000 mg (on a protein weight basis).

166. The method of embodiment 158, wherein each individual dose of the lipid binding protein-based complex administered is 800 mg to 3000 mg (on a protein weight basis).

167. The method of embodiment 158, wherein each individual dose of the lipid binding protein-based complex administered is 1000 mg to 2400 mg (on a protein weight basis).

168. The method of embodiment 158, wherein each individual dose of the lipid binding protein-based complex administered is 1000 mg to 2000 mg (on a protein weight basis).

169. The method of any one of embodiments 1 to 168, wherein the high dose of the lipid binding protein-based complex is 600 mg to 40 g (on a protein weight basis).

170. The method of any one of embodiments 1 to 168, wherein the high dose of the lipid binding protein-based complex is 3 g to 35 g (on a protein weight basis).

171. The method of any one of embodiments 1 to 168, wherein the high dose of the lipid binding protein-based complex is 5 g to 30 g (on a protein weight basis).

172. The method of any one of embodiments 1 to 171, wherein the lipid binding protein-based complex is administered by infusion.

173. The method of embodiment 172, wherein each individual dose is administered over a one to 24-hour period.

174. The method of embodiment 173, wherein each individual dose is administered over a 24-hour period.

175. The method of embodiment 172, wherein each individual dose is administered over a period of one hour or less.

176. The method of embodiment 172, wherein each individual dose is administered over a period of one-half hour to one hour.

177. The method of any one of embodiments 1 to 176, which further comprises administering an antihistamine to the subject prior to each individual dose.

178. The method of embodiment 177, wherein the antihistamine comprises dexchlorpheniramine or hydroxyzine.

179. The method of any one of embodiments 1 to 178, wherein the subject is receiving or has received one or more additional therapies and/or which further comprises administering to the subject one or more additional therapies.

180. The method of embodiment 179, wherein the one or more additional therapies comprises one or more anti-IL-6 agents.

181. The method of embodiment 180, wherein the one or more anti-IL-6 agents comprise tocilizumab, siltuximab, olokizumab, elsilimomab, BMS-945429, sirukumab, levilimab, CPSI-2364, or a combination thereof.

182. The method of embodiment 181, wherein the one or more anti-IL-6 agents comprise tocilizumab.

183. The method of any one of embodiments 179 to 182, wherein the one or more additional therapies comprise one or more antibiotics.

184. The method of embodiment 183, wherein the one or more antibiotics comprise one or more oxazolidinone class antibiotics.

185. The method of embodiment 183 or 184, wherein the one or more antibiotics comprise linezolid.

186. The method of any one of embodiments 183 to 185, wherein the one or more antibiotics comprise tedizolid.

187. The method of any one of embodiments 179 to 186, wherein the one or more additional therapies comprise one or more corticosteroids.

188. The method of embodiment 187, wherein the one or more corticosteroids comprise methylprednisolone, dexamethasone, or a combination thereof.

189. The method of any one of embodiments 179 to 188, wherein the subject has or has had a COVID-19 infection and the one or more additional therapies comprise antibodies from recovered COVID-19 patients.

190. The method of any one of embodiments 179 to 189, wherein the subject has or has had a COVID-19 infection and the one or more additional therapies comprise antibodies against the spike protein of COVID-19 (e.g., casirimivab and/or imdevimab).

191. The method of any one of embodiments 179 to 190, wherein the subject has or has had a COVID-19 infection and the one or more additional therapies comprise one or more antiviral agents.

192. The method of embodiment 191, wherein the one or more antiviral agents comprise lopinavir.

193. The method of embodiment 191 or embodiment 192, wherein the one or more antiviral agents comprise remdesivir.

194. The method of any one of embodiments 191 to 193, wherein the one or more antiviral agents comprise danoprevir.

195. The method of any one of embodiments 191 to 194, wherein the one or more antiviral agents comprise galidesivir.

196. The method of any one of embodiments 191 to 195, wherein the one or more antiviral agents comprise darunavir.

197. The method of any one of embodiments 191 to 196, wherein the one or more antiviral agents comprise ritonavir.

198. The method of any one of embodiments 179 to 197, wherein the subject has or has had a COVID-19 infection and the one or more additional therapies comprise chloroquine or hydroxychloroquine.

199. The method of any one of embodiments 179 to 198, wherein the subject has or has had a COVID-19 infection and the one or more additional therapies comprise azithromycin.

200. The method of any one of embodiments 179 to 199, wherein the subject has or has had a COVID-19 infection and the one or more additional therapies comprise an interferon.

201. The method of embodiment 200, wherein the interferon is an interferon alpha.

202. The method of embodiment 200, wherein the interferon is an interferon beta.

203. The method of any one of embodiments 200 to 202, wherein the interferon is pegylated.

204. The method of any one of embodiments 179 to 203, wherein the subject has or has had a COVID-19 infection and the one or more additional therapies comprise tacrolimus.

205. The method of any one of embodiments 179 to 204, wherein the subject has or has had a COVID-19 infection and the one or more additional therapies comprise dexamethasone.

206. The method of any one of embodiments 179 to 205, wherein the subject has or has had a COVID-19 infection and the one or more additional therapies comprise antibiotics.

207. The method of any one of embodiments 179 to 206, wherein the subject has or has had a COVID-19 infection and the one or more additional therapies comprise supplemental oxygen.

208. The method of any one of embodiments 1 to 207, wherein the lipid binding protein-based complex is CER-001.

209. The method of embodiment 208, wherein the CER-001 is a lipoprotein complex comprising ApoA-1 and phospholipids in a ApoA-1 weight:total phospholipid weight ratio of 1:2.7+/−20% and the phospholipids sphingomyelin and DPPG in a sphingomyelin:DPPG weight:weight ratio of 97:3+/−20%.

210. The method of embodiment 208, wherein the CER-001 is a lipoprotein complex comprising ApoA-1 and phospholipids in a ApoA-1 weight:total phospholipid weight ratio of 1:2.7+/−10% and the phospholipids sphingomyelin and DPPG in a sphingomyelin:DPPG weight:weight ratio of 97:3+/−10%.

211. The method of embodiment 208, wherein the CER-001 is a lipoprotein complex comprising ApoA-1 and phospholipids in a ApoA-1 weight:total phospholipid weight ratio of 1:2.7 and the phospholipids sphingomyelin and DPPG in a sphingomyelin:DPPG weight:weight ratio of 97:3.

212. The method of any one of embodiments 209 to 211, wherein the ApoA-1 has the amino acid sequence of amino acids 25-267 of SEQ ID NO:2.

213. The method of any one of embodiments 209 to 212, wherein the ApoA-1 is recombinantly expressed.

214. The method of any one of embodiments 209 to 213, wherein the CER-001 comprises natural sphingomyelin.

215. The method of embodiment 214, wherein the natural sphingomyelin is chicken egg sphingomyelin.

216. The method of any one of embodiments 209 to 213, wherein the CER-001 comprises synthetic sphingomyelin.

217. The method of embodiment 216, wherein the synthetic sphingomyelin is palmitoylsphingomyelin.

218. The method of any one of embodiments 208 to 217, wherein CER-001 is administered in the form of a formulation in which the CER-001 is at least 95% homogeneous.

219. The method of embodiment 218, wherein CER-001 is administered in the form of a formulation in which the CER-001 is at least 97% homogeneous.

220. The method of embodiment 218, wherein CER-001 is administered in the form of a formulation in which the CER-001 is at least 98% homogeneous.

221. The method of embodiment 218, wherein CER-001 is administered in the form of a formulation in which the CER-001 is at least 99% homogeneous.

222. The method of any one of embodiments 1 to 221, wherein the subject is a human.

223. The method of any one of embodiments 1 to 222, wherein the subject is not mechanically ventilated when CER-001 is administered for the first time.

224. The method of any one of embodiments 116, 118 to 207, 222, or 223, wherein the lipid binding protein-based complex is CSL-112.

225. The method of any one of embodiments 209 to 223, wherein the ApoA-1 is produced by a mammalian host cell.

226. The method of embodiment 225, wherein the mammalian host cell is a Chinese hamster ovary (CHO) cell.

227. The method of embodiment 226, wherein the CHO cell is a CHO-S cell.

228. The method of any one of embodiments 225 to 227, wherein the ApoA-1 has undergone post-translational processing (e.g., glycosylation) such that the ApoA-1 has one or more structural features (e.g., glycosylation pattern) that are different from human ApoA-1 purified from human plasma.

229. A method of treating a subject with an acute condition, comprising administering to the subject in need thereof a high dose of an Apolipoprotein A-1 (“ApoA-1”) formulation comprising ApoA-1 and one or more lipids, wherein the ApoA-1 and the lipids are in the form of lipoprotein complexes, optionally wherein the acute condition comprises acute inflammation.

230. The method of embodiment 229, wherein the high dose is administered over a period of one day to approximately two weeks, optionally wherein the high dose is administered over a period of one day, two days, three days, four days, five days, six days, seven days, eight days, nine days, 10 days, eleven days, 12 days, 13 days, 14 days or 15 days.

231. The method of embodiment 229 or embodiment 230, wherein the high dose is the aggregate of two to ten individual doses, optionally wherein the high dose is an aggregate of three, four, five, six, seven, eight, nine or 10 individual doses.

232. The method of embodiment 231, wherein a plurality of individual doses are administered daily or twice daily.

233. The method of embodiment 231 or embodiment 232, wherein a plurality of individual doses are administered two to three days apart.

234. The method of embodiment 231, wherein a plurality of individual doses are administered no more than one day apart.

235. The method of embodiment 234, which comprises administering two or more individual doses approximately 12 hours apart.

236. The method of embodiment 235, which comprises administering two individual doses approximately 12 hours apart.

237. The method of embodiment 235, which comprises administering three individual doses approximately 12 hours apart.

238. The method of embodiment 236 or embodiment 237, which further comprises administering an individual dose approximately one day later.

239. The method of embodiment 231, which comprises administering three individual doses approximately 12 hours apart and a fourth individual dose approximately one day later.

240. The method of embodiment 229, wherein the high dose is administered as a single individual dose.

241. The method of embodiment 229, wherein the high dose is the aggregate of two individual doses administered in one day.

242. The method of embodiment 241, wherein the two individual doses are administered approximately 12 hours apart.

243. The method of any one of embodiments 231 to 242, wherein each individual dose is effective to increase the subject's HDL levels.

244. The method of embodiment 243, wherein the high dose is effective to increase the subject's serum HDL levels to normal (e.g., >0.45 g/L).

245. The method of embodiment 243, wherein each individual dose is effective to increase the subject's HDL levels by at least 25%, at least 30% or at least 35% 2-4 hours after administration.

246. The method of embodiment 245, wherein each individual dose is effective to increase the subject's HDL levels by at least 25%, at least 30% or at least 35% 2 hours after administration.

247. The method of embodiment 245, wherein each individual dose is effective to increase the subject's HDL levels by at least 25%, at least 30% or at least 35% 3 hours after administration.

248. The method of embodiment 245, wherein each individual dose is effective to increase the subject's HDL levels by at least 25%, at least 30% or at least 35% 4 hours after administration.

249. The method of any one of embodiments 231 to 248, wherein each individual dose is effective to increase the subject's ApoA-1 levels.

250. The method of embodiment 249, wherein the high dose is effective increase the subject's serum ApoA-1 levels to normal (e.g., >1.1 g/L).

251. The method of embodiment 249, wherein each individual dose is effective to increase the subject's ApoA-1 levels by at least 25%, at least 30% or at least 35% 2-4 hours after administration.

252. The method of embodiment 251, wherein each individual dose is effective to increase the subject's ApoA-1 levels by at least 25%, at least 30% or at least 35% 2 hours after administration.

253. The method of embodiment 251, wherein each individual dose is effective to increase the subject's ApoA-1 levels by at least 25%, at least 30% or at least 35% 3 hours after administration.

254. The method of embodiment 251, wherein each individual dose is effective to increase the subject's ApoA-1 levels by at least 25%, at least 30% or at least 35% 4 hours after administration.

255. The method of any one of embodiments 229 to 254, wherein the high dose is effective to improve the subject's vascular endothelial function, optionally wherein vascular endothelial function is measured by circulating VCAM-1 and/or ICAM-1.

256. The method of any one of embodiments 229 to 255, wherein the high dose is effective to reduce serum levels of one or more inflammatory markers in the subject.

257. The method of embodiment 256, wherein the high dose is effective to reduce serum levels of interleukin-6 (“IL-6”).

258. The method of embodiment 256 or embodiment 257, wherein the high dose is effective to reduce serum levels of C-reactive protein.

259. The method of any one of embodiments 256 to 258, wherein the high dose is effective to reduce serum levels of D-dimer.

260. The method of any one of embodiments 256 to 259, wherein the high dose is effective to reduce serum levels of ferritin.

261. The method of any one of embodiments 256 to 260, wherein the high dose is effective to reduce serum levels of interleukin 8 (IL-8).

262. The method of any one of embodiments 256 to 260, wherein the high dose is effective to normalize serum levels of interleukin 8 (IL-8).

263. The method of any one of embodiments 256 to 262, wherein the high dose is effective to reduce serum levels of granulocyte-macrophage colony stimulating factor (GM-CSF).

264. The method of any one of embodiments 256 to 263, wherein the high dose is effective to reduce serum levels of monocyte chemoattractant protein (MCP) 1.

265. The method of any one of embodiments 256 to 264, wherein the high dose is effective to reduce serum levels of tumor necrosis factor α (TNF-α).

266. The method of any one of embodiments 256 to 265, wherein the high dose is effective to reduce serum levels of the one or more inflammatory markers from an elevated range to a normal range.

267. The method of any one of embodiments 256 to 266, wherein the high dose is effective to reduce serum levels of the one or more inflammatory markers by at least 20%, by at least 40% or by at least 60%.

268. The method of any one of embodiments 229 to 267, wherein the subject has a viral infection.

269. The method of embodiment 268, wherein the viral infection is a coronavirus infection.

270. The method of embodiment 269, wherein the coronavirus infection is COVID-19.

271. The method of any one of embodiments 229 to 270, wherein the subject has CRS or is at risk of CRS.

272. The method of embodiment 271, wherein the subject has CRS.

273. The method of embodiment 272, wherein the subject has CRS secondary to an infection.

274. The method of embodiment 273, wherein the infection is a viral infection.

275. The method of embodiment 274, wherein the viral infection is a coronavirus infection.

276. The method of embodiment 275, wherein the coronavirus is COVID-19.

277. The method of embodiment 276, wherein the viral infection is influenza infection.

278. The method of embodiment 272, wherein the subject has CRS caused by immunotherapy.

279. The method of embodiment 278, wherein the immunotherapy comprises antibody therapy.

280. The method of embodiment 278, wherein the immunotherapy comprises chimeric antigen receptor (CAR) T cell therapy.

281. The method of any one of embodiments 278 to 280, wherein the formulation is administered before the immunotherapy begins.

282. The method of any one of embodiments 278 to 281, wherein the formulation is administered concurrently with the immunotherapy.

283. The method of any one of embodiments 278 to 282, wherein the formulation is administered after the immunotherapy ends.

284. The method of embodiment 271, wherein the subject is at risk of CRS.

285. The method of embodiment 284, wherein the subject is at risk of CRS due to an infection.

286. The method of embodiment 285, wherein the infection is a viral infection.

287. The method of embodiment 286, wherein the viral infection is a coronavirus infection.

288. The method of embodiment 287, wherein the coronavirus is COVID-19.

289. The method of embodiment 286, wherein the viral infection is influenza infection.

290. The method of embodiment 284, wherein the subject is at risk of CRS due to immunotherapy.

291. The method of embodiment 290, wherein the immunotherapy comprises antibody therapy.

292. The method of embodiment 290, wherein the immunotherapy comprises chimeric antigen receptor (CAR) T cell therapy.

293. The method of any one of embodiments 290 to 292, wherein the formulation is administered before the immunotherapy begins.

294. The method of any one of embodiments 290 to 293, wherein the formulation is administered concurrently with the immunotherapy.

295. The method of any one of embodiments 290 to 294, wherein the formulation is administered after the immunotherapy ends.

296. The method of any one of embodiments 229 to 270, wherein the subject has or is at risk of developing sepsis.

297. The method of embodiment 296, wherein the sepsis is associated with a gram-negative bacterial infection.

298. The method of embodiment 296, wherein the sepsis is associated with a gram-positive bacterial infection.

299. The method of any one of embodiments 296 to 298, wherein the subject has an intra-abdominal cavity infection.

300. The method of any one of embodiments 296 to 298, wherein the subject has urosepsis.

301. The method of any one of embodiments 296 to 300, wherein the high dose is effective to reduce the severity of the sepsis.

302. The method of any one of embodiments 229 to 301, wherein the high dose is effective to reduce the likelihood that the subject will develop acute kidney injury (AKI).

303. The method of any one of embodiments 229 to 302, wherein the high dose is effective to delay the onset of AKI.

304. The method of any one of embodiments 229 to 302, wherein the high dose is effective to prevent AKI.

305. The method of any one of embodiments 229 to 301, wherein the subject has or is at risk of developing acute kidney injury (AKI).

306. The method of embodiment 305, wherein the AKI is sepsis-related AKI.

307. The method of embodiment 305, wherein the AKI is ischemia/reperfusion AKI.

308. The method of embodiment 305, wherein the AKI is cardiac surgery-associated AKI.

309. The method of embodiment 305, wherein the AKI is hepatorenal syndrome (HRS) AKI.

310. The method of embodiment 309, wherein the HRS is type 1 HRS.

311. The method of embodiment 309, wherein the HRS is type 2 HRS.

312. The method of any one of embodiments 305 to 311, wherein the subject has AKI.

313. The method of embodiment 312, wherein the AKI is secondary to a viral infection, optionally wherein the viral infection is COVID-19.

314. The method of embodiment 312 or embodiment 313, wherein the high dose is effective to reduce the severity of the AKI.

315. The method of any one of embodiments 305 to 311, wherein the subject is at risk for AKI.

316. The method of embodiment 315, wherein the subject has sepsis.

317. The method of embodiment 316, wherein the sepsis is associated with a gram-negative bacterial infection.

318. The method of embodiment 316, wherein the sepsis is associated with a gram-positive bacterial infection.

319. The method of any one of embodiments 316 to 318, wherein the subject has an intra-abdominal cavity infection.

320. The method of any one of embodiments 316 to 318, wherein the subject has urosepsis.

321. The method of embodiment 315, wherein the subject has a viral infection, optionally wherein the viral infection is COVID-19.

322. The method of embodiment 315, wherein the subject has undergone cardiac surgery.

323. The method of embodiment 315, wherein the subject has acute liver disease.

324. The method of embodiment 315, wherein the subject has chronic liver disease.

325. The method of any one of embodiments 315 to 324, wherein the high dose is effective to reduce the likelihood that the subject will develop AKI.

326. The method of any one of embodiments 315 to 325, wherein the high dose is effective to delay the onset of AKI.

327. The method of any one of embodiments 315 to 325, wherein the high dose is effective to prevent AKI.

328. The method of any one of embodiments 315 to 326, wherein if the subject develops AKI, the high dose is effective to reduce the severity of the AKI.

329. The method of any one of embodiments 296 to 328, wherein the subject has a total SOFA score of 2 to 24 prior to administration of the formulation.

330. The method of embodiment 329, wherein the subject has a total SOFA score of 2 to 5 prior to administration of the formulation.

331. The method of embodiment 329, wherein the subject has a total SOFA score of 2 to 10 prior to administration of the formulation.

332. The method of embodiment 329, wherein the subject has a total SOFA score of 5 to 15 prior to administration of the formulation.

333. The method of embodiment 329, wherein the subject has a total SOFA score of 8 to 13 prior to administration of the formulation

334. The method of embodiment 329, wherein the subject has a total SOFA score of 10 to 15 prior to administration of the formulation.

335. The method of embodiment 329, wherein the subject has a total SOFA score of 10 to 20 prior to administration of the formulation.

336. The method of embodiment 329, wherein the subject has a total SOFA score of 15 to 24 prior to administration of the formulation.

337. The method of any one of embodiments 229 to 336, wherein the subject has an endotoxin activity level of >0.6 prior to administration of the formulation.

338. The method of any one of embodiments 229 to 337, wherein the high dose is effective to reduce the subject's endotoxin activity level.

339. The method of any one of embodiments 229 to 338, wherein the formulation is a reconstituted HDL or HDL mimetic.

340. The method of any one of embodiments 229 to 339, wherein the formulation comprises a sphingomyelin.

341. The method of any one of embodiments 229 to 340, wherein the formulation comprises a negatively charged lipid.

342. The method of embodiment 341, wherein the negatively charged lipid is 1,2-dipalmitoyl-sn-glycero-3-[phospho-rac-(1-glycerol) (DPPG) or a salt thereof.

343. The method of any one of embodiments 229 to 342, wherein the formulation is administered systemically, optionally by infusion.

344. The method of any one of embodiments 229 to 343, wherein the formulation is administered until serum levels of one or more inflammatory markers are reduced.

345. The method of embodiment 344, wherein the formulation is administered until serum levels of one or more inflammatory markers are reduced to a normal range(s).

346. The method of embodiment 344, wherein the formulation is administered until serum levels of one or more inflammatory markers are reduced below a baseline level(s) for the one or more inflammatory markers measured prior to lipid binding protein-based complex administration.

347. The method of any one of embodiments 229 to 346, wherein each individual dose of the formulation administered is 4-40 mg/kg (on a protein weight basis).

348. The method of embodiment 347, wherein each individual dose of the formulation is 4-30 mg/kg (on a protein weight basis).

349. The method of embodiment 347, wherein each individual dose of the formulation is 15-25 mg/kg (on a protein weight basis).

350. The method of embodiment 347, wherein each individual dose of the formulation is 10-30 mg/kg (on a protein weight basis).

351. The method of embodiment 347, wherein each individual dose of the formulation is 10-20 mg/kg (on a protein weight basis).

352. The method of embodiment 347, wherein each individual dose of the formulation is 5 mg/kg (on a protein weight basis).

353. The method of embodiment 347, wherein each individual dose of the formulation is 10 mg/kg (on a protein weight basis).

354. The method of embodiment 347, wherein each individual dose of the formulation is 15 mg/kg (on a protein weight basis).

355. The method of embodiment 347, wherein each individual dose of the formulation is 20 mg/kg (on a protein weight basis).

356. The method of embodiment 347, wherein each individual dose of the formulation is 5 to 15 mg/kg (on a protein weight basis).

357. The method of embodiment 347, wherein each individual dose of the formulation is 10 to 20 mg/kg (on a protein weight basis).

358. The method of embodiment 347, wherein each individual dose of the formulation is 15 to 25 mg/kg (on a protein weight basis).

359. The method of any one of embodiments 229 to 358, wherein the high dose is administered according to an induction regimen, optionally followed by a consolidation regimen.

360. The method of embodiment 359, wherein the induction regimen comprises administering the formulation once daily or twice daily.

361. The method of embodiment 359 or embodiment 360, wherein the consolidation regimen comprises administering the formulation once daily or once every two days.

362. The method of any one of embodiments 229 to 361, wherein the subject is not treated with a maintenance regimen.

363. The method of any one of embodiments embodiment 359 to 362, wherein the consolidation regimen comprises administering one or more doses of the formulation to the subject one or more days after administration of the final dose of the induction regimen.

364. The method of embodiment 363, wherein the first dose of the formulation administered during the consolidation regimen is administered two or more days after administration of the final dose of the induction regimen.

365. The method of embodiment 363, wherein the first dose of the formulation administered during the consolidation regimen is administered three or more days after administration of the final dose of the induction regimen.

366. The method of embodiment 365, wherein the first dose of the formulation administered during the consolidation regimen is administered three days after administration of the final dose of the induction regimen.

367. The method of any one of embodiments 359 to 366, which comprises an induction regimen comprising twice daily administration of the formulation on days 1, 2, and 3 and a consolidation regimen comprising two doses of the formulation on day 6.

368. The method of any one of embodiments 359 to 367, wherein each individual dose of the formulation administered in the induction regimen is 4-40 mg/kg (on a protein weight basis).

369. The method of any one of embodiments 359 to 368, wherein each individual dose of the formulation administered in the induction regimen is 4-30 mg/kg (on a protein weight basis).

370. The method of any one of embodiments 359 to 368, wherein each individual dose of the formulation administered in the induction regimen is 15-25 mg/kg (on a protein weight basis).

371. The method of any one of embodiments 359 to 368, wherein each individual dose of the formulation administered in the induction regimen is 10-30 mg/kg (on a protein weight basis).

372. The method of any one of embodiments 359 to 368, wherein each individual dose of the formulation administered in the induction regimen is 10-20 mg/kg (on a protein weight basis).

373. The method of any one of embodiments 359 to 368, wherein each individual dose of the formulation administered in the induction regimen is 5 mg/kg (on a protein weight basis).

374. The method of any one of embodiments 359 to 368, wherein each individual dose of the formulation administered in the induction regimen is 10 mg/kg (on a protein weight basis).

375. The method of any one of embodiments 359 to 368, wherein each individual dose of the formulation administered in the induction regimen is 15 mg/kg (on a protein weight basis).

376. The method of any one of embodiments 359 to 368, wherein each individual dose of the formulation administered in the induction regimen is 20 mg/kg (on a protein weight basis).

377. The method of any one of embodiments 359 to 376, wherein the dose of the formulation administered in the consolidation regimen is 5 to 15 mg/kg (on a protein weight basis).

378. The method of any one of embodiments 359 to 376, wherein the dose of the formulation administered in the consolidation regimen is 10 to 20 mg/kg (on a protein weight basis).

379. The method of any one of embodiments 359 to 376, wherein the dose of the formulation administered in the consolidation regimen is 15 to 25 mg/kg (on a protein weight basis).

380. The method of any one of embodiments 359 to 376, wherein the dose of the formulation administered in the consolidation regimen is 5 mg/kg (on a protein weight basis).

381. The method of any one of embodiments 359 to 376, wherein the dose of the formulation administered in the consolidation regimen is 10 mg/kg (on a protein weight basis).

382. The method of any one of embodiments 359 to 376, wherein the dose of the formulation administered in the consolidation regimen is 15 mg/kg (on a protein weight basis).

383. The method of any one of embodiments 229 to 382, wherein each individual dose of the formulation administered is 300 mg to 4000 mg (on a protein weight basis).

384. The method of embodiment 383, wherein each individual dose of the formulation administered is 300 mg to 3000 mg (on a protein weight basis).

385. The method of embodiment 383, wherein each individual dose of the formulation administered is 300 mg to 1500 mg (on a protein weight basis).

386. The method of embodiment 383, wherein each individual dose of the formulation administered is 400 mg to 4000 mg (on a protein weight basis).

387. The method of embodiment 383, wherein each individual dose of the formulation administered is 400 mg to 1500 mg (on a protein weight basis).

388. The method of embodiment 383, wherein each individual dose of the formulation administered is 500 mg to 1200 mg (on a protein weight basis).

389. The method of embodiment 383, wherein each individual dose of the formulation administered is 500 mg to 1000 mg (on a protein weight basis).

390. The method of embodiment 383, wherein each individual dose of the formulation administered is 600 mg to 3000 mg (on a protein weight basis).

391. The method of embodiment 383, wherein each individual dose of the formulation administered is 800 mg to 3000 mg (on a protein weight basis).

392. The method of embodiment 383, wherein each individual dose of the formulation administered is 1000 mg to 2400 mg (on a protein weight basis).

393. The method of embodiment 383, wherein each individual dose of the formulation administered is 1000 mg to 2000 mg (on a protein weight basis).

394. The method of any one of embodiments 229 to 393, wherein the high dose of the formulation is 600 mg to 40 g (on a protein weight basis).

395. The method of any one of embodiments 229 to 393, wherein the high dose of the formulation is 3 g to 35 g (on a protein weight basis).

396. The method of any one of embodiments 229 to 393, wherein the high dose of the formulation is 5 g to 30 g (on a protein weight basis).

397. The method of any one of embodiments 229 to 396, wherein the formulation is administered by infusion.

398. The method of embodiment 397, wherein each individual dose is administered over a one to 24-hour period.

399. The method of embodiment 398, wherein each individual dose is administered over a 24-hour period.

400. The method of embodiment 397, wherein each individual dose is administered over a period of one hour or less.

401. The method of embodiment 397, wherein each individual dose is administered over a period of one-half hour to one hour.

402. The method of any one of embodiments 229 to 401, which further comprises administering an antihistamine to the subject prior to each individual dose.

403. The method of embodiment 402, wherein the antihistamine comprises dexchlorpheniramine or hydroxyzine.

404. The method of any one of embodiments 229 to 403, wherein the subject is receiving or has received one or more additional therapies and/or which further comprises administering to the subject one or more additional therapies.

405. The method of embodiment 404, wherein the one or more additional therapies comprises one or more anti-IL-6 agents.

406. The method of embodiment 405, wherein the one or more anti-IL-6 agents comprise tocilizumab, siltuximab, olokizumab, elsilimomab, BMS-945429, sirukumab, levilimab, CPSI-2364, or a combination thereof.

407. The method of embodiment 406, wherein the one or more anti-IL-6 agents comprise tocilizumab.

408. The method of any one of embodiments 404 to 407, wherein the one or more additional therapies comprise one or more antibiotics.

409. The method of embodiment 408, wherein the one or more antibiotics comprise one or more oxazolidinone class antibiotics.

410. The method of embodiment 408 or embodiment 409, wherein the one or more antibiotics comprise linezolid.

411. The method of any one of embodiments 408 to 410, wherein the one or more antibiotics comprise tedizolid.

412. The method of any one of embodiments 404 to 410, wherein the one or more additional therapies comprise one or more corticosteroids.

413. The method of embodiment 412, wherein the one or more corticosteroids comprise methylprednisolone, dexamethasone, or a combination thereof.

414. The method of any one of embodiments 404 to 413, wherein the subject has or has had a COVID-19 infection and the one or more additional therapies comprise antibodies from recovered COVID-19 patients.

415. The method of any one of embodiments 404 to 414, wherein the subject has or has had a COVID-19 infection and the one or more additional therapies comprise antibodies against the spike protein of COVID-19 (e.g., casirimivab and/or imdevimab).

416. The method of any one of embodiments 404 to 415, wherein the subject has or has had a COVID-19 infection and the one or more additional therapies comprise one or more antiviral agents.

417. The method of embodiment 416, wherein the one or more antiviral agents comprise lopinavir.

418. The method of embodiment 416 or embodiment 417, wherein the one or more antiviral agents comprise remdesivir.

419. The method of any one of embodiments 416 to 418, wherein the one or more antiviral agents comprise danoprevir.

420. The method of any one of embodiments 416 to 419, wherein the one or more antiviral agents comprise galidesivir.

421. The method of any one of embodiments 416 to 420, wherein the one or more antiviral agents comprise darunavir.

422. The method of any one of embodiments 416 to 421, wherein the one or more antiviral agents comprise ritonavir.

423. The method of any one of embodiments 416 to 422, wherein the subject has or has had a COVID-19 infection and the one or more additional therapies comprise chloroquine or hydroxychloroquine.

424. The method of any one of embodiments 404 to 423, wherein the subject has or has had a COVID-19 infection and the one or more additional therapies comprise azithromycin.

425. The method of any one of embodiments 404 to 424, wherein the subject has or has had a COVID-19 infection and the one or more additional therapies comprise an interferon.

426. The method of embodiment 425, wherein the interferon is an interferon alpha.

427. The method of embodiment 425, wherein the interferon is an interferon beta.

428. The method of any one of embodiments 425 to 427, wherein the interferon is pegylated.

429. The method of any one of embodiments 404 to 428, wherein the subject has or has had a COVID-19 infection and the one or more additional therapies comprise tacrolimus.

430. The method of any one of embodiments 404 to 429, wherein the subject has or has had a COVID-19 infection and the one or more additional therapies comprise dexamethasone.

431. The method of any one of embodiments 404 to 430, wherein the subject has or has had a COVID-19 infection and the one or more additional therapies comprise antibiotics.

432. The method of any one of embodiments 404 to 431, wherein the subject has or has had a COVID-19 infection and the one or more additional therapies comprise supplemental oxygen.

433. The method of embodiment 432, wherein the formulation comprises the ApoA-1 and phospholipids in an ApoA-1 weight:total phospholipid weight ratio of 1:2.7+/−20% and the phospholipids sphingomyelin and DPPG in a sphingomyelin:DPPG weight:weight ratio of 97:3+/−20%.

434. The method of embodiment 432, wherein the formulation comprises the ApoA-1 and phospholipids in an ApoA-1 weight:total phospholipid weight ratio of 1:2.7+/−10% and the phospholipids sphingomyelin and DPPG in a sphingomyelin:DPPG weight:weight ratio of 97:3+/−10%.

435. The method of embodiment 432, wherein the formulation comprises the ApoA-1 and phospholipids in an ApoA-1 weight:total phospholipid weight ratio of 1:2.7 and the phospholipids sphingomyelin and DPPG in a sphingomyelin:DPPG weight:weight ratio of 97:3.

436. The method of any one of embodiments 433 to 435, wherein the ApoA-1 has the amino acid sequence of amino acids 25-267 of SEQ ID NO:2.

437. The method of any one of embodiments 433 to 436, wherein the ApoA-1 is recombinantly expressed.

438. The method of any one of embodiments 433 to 437, wherein the formulation comprises natural sphingomyelin.

439. The method of embodiment 438, wherein the natural sphingomyelin is chicken egg sphingomyelin.

440. The method of any one of embodiments 433 to 437, wherein the formulation comprises synthetic sphingomyelin.

441. The method of embodiment 440, wherein the synthetic sphingomyelin is palmitoylsphingomyelin.

442. The method of any one of embodiments 433 to 441, wherein the formulation is at least 95% homogeneous.

443. The method of embodiment 442, wherein the formulation is at least 97% homogeneous.

444. The method of embodiment 442, wherein the formulation is at least 98% homogeneous.

445. The method of embodiment 442, wherein the formulation is at least 99% homogeneous.

446. The method of any one of embodiments 443 to 445, wherein the subject is a human.

447. The method of any one of embodiments 443 to 446, wherein the subject is not mechanically ventilated when the formulation is administered for the first time.

448. The method of any one of embodiments 437 to 447, wherein the ApoA-1 is produced by a mammalian host cell.

449. The method of embodiment 448, wherein the mammalian host cell is a Chinese hamster ovary (CHO) cell.

450. The method of embodiment 449, wherein the CHO cell is a CHO-S cell.

451. The method of any one of embodiments 448 to 450, wherein the ApoA-1 has undergone post-translational processing (e.g., glycosylation) such that the ApoA-1 has one or more structural features (e.g., glycosylation pattern) that are different from human ApoA-1 purified from human plasma.

While various specific embodiments have been illustrated and described, it will be appreciated that various changes can be made without departing from the spirit and scope of the disclosure(s)

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

Any discussion of documents, acts, materials, devices, articles or the like that has been included in this specification is solely for the purpose of providing a context for the present disclosure. It is not to be taken as an admission that any or all of these matters form part of the prior art base or were common general knowledge in the field relevant to the present disclosure as it existed anywhere before the priority date of this application.

What is claimed is:

1. A lipid binding protein-based complex for use in a method of treating an acute condition, wherein the method comprises administering a high dose of the lipid binding protein-based complex to a human subject in need thereof in a single dose or a plurality of individual doses administered no more than one day apart, optionally wherein the acute condition comprises acute inflammation.

2. The lipid binding protein-based complex for use according to claim 1, wherein the high dose is the aggregate of two to four individual doses, optionally wherein the high dose is an aggregate of two, three, or four individual doses.

3. The lipid binding protein-based complex for use according to claim 2, which comprises administering two or more individual doses approximately 12 hours apart.

4. The lipid binding protein-based complex for use according to claim 3, which comprises administering two individual doses approximately 12 hours apart.

5. The lipid binding protein-based complex for use according to claim 3, which comprises administering three individual doses approximately 12 hours apart.

6. The lipid binding protein-based complex for use according to claim 4 or claim 5, which further comprises administering an individual dose approximately one day later.

7. The lipid binding protein-based complex for use according to claim 2, which comprises administering three individual doses approximately 12 hours apart and a fourth individual dose approximately one day later.

8. The lipid binding protein-based complex for use according to claim 1, wherein the high dose is administered as a single individual dose.

9. The lipid binding protein-based complex for use according to claim 1, wherein the high dose is the aggregate of two individual doses administered in one day.

10. The lipid binding protein-based complex for use according to claim 9, wherein the two individual doses are administered approximately 12 hours apart.

11. The lipid binding protein-based complex for use according to any one of claims 1 to 10, wherein the high dose is effective to increase the subject's serum HDL levels, optionally wherein the high dose is effective to increase the subject's serum HDL levels to normal.

12. The lipid binding protein-based complex for use according to any one of claims 1 to 11, wherein the high dose is effective to increase the subject's serum ApoA-1 levels, optionally wherein the high dose is effective to increase the subject's serum ApoA-1 levels to normal.

13. The lipid binding protein-based complex for use according to any one of claims 1 to 12, wherein the high dose is effective to normalize serum levels of interleukin 8 (IL-8).

14. The lipid binding protein-based complex for use according to any one of claims 1 to 13, wherein the subject has a viral infection.

15. The lipid binding protein-based complex for use according to claim 14, wherein the viral infection is a coronavirus infection.

16. The lipid binding protein-based complex for use according to claim 15, wherein the coronavirus infection is COVID-19.

17. The lipid binding protein-based complex for use according to any one of claims 1 to 16, wherein the subject has CRS or is at risk of CRS.

18. The lipid binding protein-based complex for use according to claim 17, wherein the subject is at risk of CRS.

19. The lipid binding protein-based complex for use according to claim 18, wherein the subject is at risk of CRS due to an infection.

20. The lipid binding protein-based complex for use according to claim 19, wherein the infection is a viral infection.

21. The lipid binding protein-based complex for use according to claim 20, wherein the viral infection is a coronavirus infection.

22. The lipid binding protein-based complex for use according to claim 21, wherein the coronavirus is COVID-19.

23. The lipid binding protein-based complex for use according to any one of claims 1 to 16, wherein the subject has or is at risk of developing sepsis.

24. The lipid binding protein-based complex for use according to claim 23, wherein the high dose is effective to reduce the severity of the sepsis.

25. The lipid binding protein-based complex for use according to any one of claims 1 to 24, wherein the high dose is effective to reduce the likelihood that the subject will develop acute kidney injury (AKI).

26. The lipid binding protein-based complex for use according to any one of claims 1 to 25, wherein the high dose is effective to delay the onset of AKI.

27. The lipid binding protein-based complex for use according to any one of claims 1 to 25, wherein the high dose is effective to prevent AKI.

28. The lipid binding protein-based complex for use according to any one of claims 1 to 24, wherein the subject has or is at risk of developing acute kidney injury (AKI).

29. The lipid binding protein-based complex for use according to claim 28, wherein the AKI is sepsis-related AKI.

30. The lipid binding protein-based complex for use according to claim 28, wherein the AKI is ischemia/reperfusion AKI.

31. The lipid binding protein-based complex for use according to claim 28, wherein the AKI is cardiac surgery-associated (CSA) AKI.

32. The lipid binding protein-based complex for use according to claim 28, wherein the AKI is hepatorenal syndrome (HRS) AKI.

33. The lipid binding protein-based complex for use according to any one of claims 28 to 32, wherein the subject has AKI.

34. The lipid binding protein-based complex for use according to claim 33, wherein the high dose is effective to reduce the severity of the AKI.

35. The lipid binding protein-based complex for use according to any one of claims 28 to 32, wherein the subject is at risk for AKI.

36. The lipid binding protein-based complex for use according to claim 35, wherein the high dose is effective to reduce the likelihood that the subject will develop AKI.

37. The lipid binding protein-based complex for use according to claim 35 or claim 36, wherein the high dose is effective to delay the onset of AKI.

38. The lipid binding protein-based complex for use according to claim 35 or claim 36, wherein the high dose is effective to prevent AKI.

39. The lipid binding protein-based complex for use according to any one of claims 35 to 37, wherein if the subject develops AKI, the high dose is effective to reduce the severity of the AKI.

40. The lipid binding protein-based complex for use according to any one of claims 1 to 39, wherein the lipid binding protein-based complex is a reconstituted HDL or HDL mimetic.

41. The lipid binding protein-based complex for use according to any one of claims 1 to 39, wherein the lipid binding protein-based complex is an Apomer or a Cargomer.

42. The lipid binding protein-based complex for use according to any one of claims 1 to 41, wherein the lipid binding protein-based complex comprises a sphingomyelin.

43. The lipid binding protein-based complex for use according to any one of claims 1 to 42, wherein the lipid binding protein-based complex comprises a negatively charged lipid.

44. The lipid binding protein-based complex for use according to claim 43, wherein the negatively charged lipid is 1,2-dipalmitoyl-sn-glycero-3-[phospho-rac-(1-glycerol) (DPPG) or a salt thereof.

45. The lipid binding protein-based complex for use according to claim 40, wherein the lipid binding protein-based complex is CER-001, CSL-111, CSL-112, CER-522 or ETC-216.

46. The lipid binding protein-based complex for use according to claim 45, wherein the lipid binding protein-based complex is CER-001.

47. The lipid binding protein-based complex for use according to claim 45, wherein the lipid binding protein-based complex is CSL-112.

48. The lipid binding protein-based complex for use according to any one of claims 1 to 47, wherein the method comprises administering the lipid binding protein-based systemically, optionally by infusion.

49. The lipid binding protein-based complex for use according to any one of claims 1 to 48, wherein the method comprises administering the lipid binding protein-based complex over a period of one hour or less.

50. The lipid binding protein-based complex for use according to any one of claims 1 to 49, wherein the method comprises administering the lipid binding protein-based complex over a period of one-half hour to one hour.

51. The lipid binding protein-based complex for use according to any one of claims 1 to 50, wherein each individual dose of the lipid binding protein-based complex administered is 4-40 mg/kg (on a protein weight basis).

52. The lipid binding protein-based complex for use according to claim 51, wherein each individual dose of the lipid binding protein-based complex administered is 10 mg/kg (on a protein weight basis).

53. The lipid binding protein-based complex for use according to claim 51, wherein each individual dose of the lipid binding protein-based complex administered is 15 mg/kg (on a protein weight basis).

54. The lipid binding protein-based complex for use according to any one of claims 1 to 53, wherein the method further comprises administering an antihistamine to the subject prior to each individual dose.

55. The lipid binding protein-based complex for use according to of any one of claims 1 to 54, wherein the subject is receiving or has received one or more additional therapies and/or wherein the method further comprises administering to the subject one or more additional therapies.

56. An Apolipoprotein A-1 (“ApoA-1”) formulation comprising ApoA-1 and one or more lipids wherein the ApoA-1 and the lipids are in the form of lipoprotein complexes for use in a method of treating an acute condition, wherein the method comprises administering a high dose of the formulation to a human subject in need thereof in a single dose or a plurality of individual doses administered no more than one day apart, optionally wherein the acute condition comprises acute inflammation.


SEQUENCE LISTING (2 sequences)

SEQ ID NO: 1 - protein, 241 aa, synthetic construct
PPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 267 aa, synthetic construct
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ